(19) United States
(12) Patent Application Publication
(10) Pub. No.: US 2025/0051760 A1
(43) Pub. Date: Feb. 13, 2025

(54) SOLID-PHASE SCREENING FOR HIGH-PERFORMING BACTERIAL STRAINS

(71) Applicant: ABSCI CORPORATION, Vancouver, WA (US)

(72) Inventor: Jia Liu, Vancouver, WA (US)

(21) Appl. No.: 18/719,906

(22) PCT Filed: Dec. 16, 2022
(86) PCT No.: PCT/US2022/053107

§ 371 Date: Jun. 14, 2024

Related U.S. Application Data
(60) Provisional application No. 63/291,059, filed on Dec. 17, 2021.

(51) Int. Cl. C12N 15/10

(52) U.S. Cl. CPC C12N 15/1086; C12N 15/1072

(57) ABSTRACT

The present disclosure provides compositions and methods for identifying a host cell or host cells from a population of cells that produce a gene product of interest. The present disclosure further provides a solid-phase screening workflow that enables affinity determination and the identification of high-producing strains.

### CROSS-REFERENCE TO RELATED APPLICATIONS

The present application claims priority to U.S. Provisional Patent Application No. 63/291,059, filed on Dec. 17, 2021, their entirety of which are incorporated by reference herein.

### BACKGROUND

De novo antibody discovery, where the goal is to produce a high affinity antibody to a known antigen, has historically been performed by immunizing animals such as rabbits or mice, and eliciting an immune response in the host to the antigen. After multiple rounds of immunization, the serum is tested to determine the antibody titer to the antigen. For monoclonal antibodies, at the end of several rounds of immunization, the B-cell population from the host spleen is recovered, expanded clonally in vitro, and the secreted antibody is characterized to determine affinity. Contemporary methods offer workflows for de novo antibody discovery without the use of animals. Such methods rely on the creation of large, diverse “libraries” of antibody or antibody fragment capable of binding the antigen and screening the library to identify the highest affinity members for subsequent rounds of in vitro “maturation” to obtain higher binding affinity variants. Commonly used library formats for in vitro de novo antibody discovery typically produce antibody fragments such as (1) single chain variable domain (scFv), (2) Fab, or (3) nanobodies. The antibody fragments are typically expressed (displayed) on bacteriophage surface coat or, for a subset of the antibody fragments, can be expressed on the E. coli surface as a fusion protein using surface display technology. The phage or E. coli surface display library would be subjected to multiple rounds of counter selection/selection using the antigen for affinity capture of phage or E. coli to a solid support surface. After washing to enrich for the subset of phage or E. coli bearing the highest affinity antibody fragment immobilized to the solid support surface, the bound phage or E. coli is eluted and analyzed to determine binding affinity.

Current methodologies suffer from several problems, including: (1) The library of antibody fragment must use either phage display or E. coli surface display-no intracellular/cytoplasmic antibody fragment can be screened; (2) Selection is not amendable to full antibody molecules (mAb) because such an entity cannot be displayed for screening; (3) The number of independent clones of phage or E. coli in the library that represents the subset with the best binding affinity cannot be determined until at the very last step where sequencing and measuring the binding affinity are determined separately for each isolated clone; and (4) The current methodology does not allow one to determine if the antibody fragment library contains any modest to high binding affinity clone (Dissociation constant<70 Kd) until the very end, where the affinity of each recovered clone is measured independently.

The present disclosure addresses these needs and can be applied to screen any microbial expression library with a suitable probe.

### SUMMARY

In one embodiment, the present disclosure provides a method of identifying a host cell that produces a gene product of interest, said method comprising the steps of: (a) plating a population of host cells on a growth substrate, wherein said host cells comprise an expression construct encoding one or more gene products, and incubating the host cells under conditions that allow host cell growth on the growth substrate; (b) preparing at least one replica plate and incubating said replica plate under conditions that allow host cell growth and production of the gene product; (c) transferring host cells from the at the least one replica plate of (b) to a membrane; (d) preparing the host cells that have been transferred to the membrane in (c) for probing, comprising (i) optionally fixing the host cells under conditions that allow immobilization of cellular components; (ii) blocking the host cells; and (iii) optionally lysing the host cells under conditions that allow permeabilization of the host cells; (e) contacting the permeabilized host cells with a probe solution comprising at least one probe under conditions that allow binding of the at least one probe to a gene product of interest, and thereby forming a probe-gene product complex; and (f) imaging the host cells under conditions that allow identifying the host cell that produces the gene product of interest. In another embodiment, step (c) further comprises marking the at least one replica plate and membrane to allow spatial alignment of the at least one replica plate and membrane.

In still other embodiments, an aforementioned method is provided wherein the host cell is selected from the group consisting of a eukaryotic cell, a prokaryotic cell, a bacterial cell, a mammalian cell and an insect cell. In still other embodiments, an aforementioned method is provided wherein the gene product of interest is selected from the group consisting of a therapeutic protein, an antibody, a Fab, a scFv, a nanobody, a T cell receptor, and a chimeric antigen receptor, or fragments thereof. In one embodiment, the gene product of interest is an intracellular protein.

In yet other embodiments, an aforementioned method is provided wherein the population of host cells comprises approximately one thousand to approximately one billion host cells. In other embodiments, the population of host cells have been modified to produce a gene product from an expression construct. In one embodiment, the expression construct comprises two or more inducible promoters. In still another embodiment, at least one inducible promoter is a propionate-inducible promoter and at least one other inducible promoter is an L-arabinose-inducible promoter.

The present disclosure also provides, in some embodiments, an aforementioned method wherein the host cells have been genetically modified to comprise one or more of: (a) an alteration of gene function of at least one gene encoding a transporter protein for an inducer of at least one inducible promoter; (b) a reduced level of gene function of at least one gene encoding a protein that metabolizes an inducer of at least one inducible promoter; (c) a reduced level of gene function of at least one gene encoding a protein involved in biosynthesis of an inducer of at least one inducible promoter; (d) an altered gene function of a gene that affects the reduction/oxidation environment of the host cell cytoplasm; (e) a reduced level of gene function of a gene that encodes a reductase; (f) at least one expression construct encoding at least one disulfide bond isomerase protein; (g) at least one polynucleotide encoding a form of DsbC lacking a signal peptide; and/or (h) at least one polynucleotide encoding Ervlp.

In still other embodiments, an aforementioned method is provided wherein 2, 3, 4, 5 or more replica plates are prepared. In one embodiment, the replica plates contain an antibiotic and at least one inducers of expression of the gene product of interest. In still other embodiments, the membrane is selected from the group consisting of a nitrocellulose membrane and a PVDF (polyvinylidene fluoride) membrane. In other embodiments, the replica plate and membrane of step (c) are contacted with a colored substance. In one embodiment, the colored substance is selected from the group consisting of acrylic paint and a dye. In another embodiment, the colored substance is contacted to the plate and membrane with an instrument selected from the group consisting of a needle and a pen.

In other embodiments, an aforementioned method is provided wherein the conditions that allow immobilization of cellular components comprise contacting the membrane with a composition comprising one or more of glutaraldehyde and paraformaldehyde. In other embodiments, the conditions that allow permeabilization of the host cells comprise contacting the membrane with a composition comprising one or more of lysozyme and EDTA.

In still other embodiments, an aforementioned method is provided wherein the at least one probe is selected from the group consisting of an antibody or functional fragment thereof, a nucleic acid-binding protein or functional fragment thereof, a receptor or functional fragment thereof, a ligand or functional fragment thereof, and antigen or functional fragment thereof, and a peptide or polypeptide capable of being bound by the gene product of interest. In one embodiment, the probe comprises a reporter moiety selected from the group consisting of biotin, a histidine tag, a Fc tag, a spy tag, a Strp tag, and an Avi tag.

The present disclosure also provides, in some embodiments, an aforementioned method wherein, when 2, 3, 4, 5 or more replica plates have been prepared, each membrane prepared from the replica plates is contacted with a different concentration of the probe solution. In still other embodiments, an aforementioned method is provided wherein the imaging comprises contacting the membrane with a composition comprising an activator of the reporter moiety. In another embodiment, the contacting the membrane with a composition comprising an activator of the reporter moiety is repeated once, twice, or three or more times. In still another embodiment, the activator is selected from the group consisting of alkaline phosphatase, streptavidin alkaline phosphatase, and streptavidin alkaline phosphatase dextran polymer.

In still other embodiments, an aforementioned method is provided wherein the imaging comprises a method selected from the group consisting of chemiluminescence, radiography, fluorescence and colorimetric analyses. In still other embodiments, an aforementioned is provided further comprising the step of re-plating one or more host cells that have been identified as capable of producing the gene product of interest. In another embodiment, re-plated host cell is subjected to the method of claim 1 to confirm the host cell's capability to produce the gene product of interest and/or to isolate the host cell strain from other host cell strains that have been identified as capable of producing the gene product of interest. In some embodiments the methods may further comprise determining the affinity of the probe-gene product complex.

The present disclosure also provides, in one embodiment, a method of screening a host cell from a population of host cells that produces a gene product of interest, said method comprising the steps of: (a) plating a population of host cells on a growth substrate, wherein said host cells comprise an expression construct encoding one or more gene products, and incubating the host cells under conditions that allow host cell growth on the growth substrate; (b) preparing at least one replica plate and incubating said replica plate under conditions that allow host cell growth and production of the gene product; (c) transferring host cells from the at the least one replica plate of (b) to a membrane; (d) preparing the host cells that have been transferred to the membrane in (c) for probing, comprising (i) fixing the host cells under conditions that allow immobilization of cellular components; (ii) blocking the host cells; and (iii) optionally lysing the host cells under conditions that allow permeabilization of the host cells; (e) contacting the permeabilized host cells with a probe solution comprising at least one probe under conditions that allow binding of the at least one probe to a gene product of interest, and thereby forming a probe-gene product complex; and (f) imaging the host cells under conditions that allow identifying the host cell that produces the gene product of interest.

In still another embodiment, the present disclosure provides a method of determining the relative affinity of a probe-gene product of interest, said method comprising the steps of: (a) plating a population of host cells on a growth substrate, wherein said host cells comprise an expression construct encoding one or more gene products, and incubating the host cells under conditions that allow host cell growth on the growth substrate; (b) preparing at least three replica plates and incubating said replica plate under conditions that allow host cell growth and production of the gene product; (c) transferring host cells from the at the least three replica plates of (b) to separate membranes; (d) preparing the host cells that have been transferred to the membrane in (c) for probing, comprising (i) fixing the host cells under conditions that allow immobilization of cellular components; (ii) blocking the host cells; and (iii) optionally lysing the host cells under conditions that allow permeabilization of the host cells; (e) contacting the permeabilized host cells on the at least three replica membranes with a probe solution comprising at least one probe under conditions that allow binding of the at least one probe to a gene product of interest, and thereby forming a probe-gene product complex, and wherein the probe solution comprises a different probe concentration; and (f) imaging the host cells under conditions that allow determining the relative affinity of a probe-gene product of interest.

### BRIEF DESCRIPTION OF THE DRAWINGS

FIG. 1 shows a solid-phase screening workflow.

FIG. 2 shows that good signal fidelity was obtained in two replica membranes as the positive spots are exactly superimposable. The left replicate membrane was probed at high (5.0 nM) and the right replicate membrane at low (0.5 nM, right) concentrations of antigen (Her2). There were ˜10,000 colonies on the original source plate that represents a cytoplasmicly expressed VHH antibody fragment library.

FIG. 3 shows that the solid-phase assay correctly rank ordered three Fab variants by Her2 binding affinity using a three-concentration (5.0, 0.5, 0.05 nM) Her2 binding response. The four E. coli strains and the Her2 binding affinities (Kd) are: (1) wild type Trastuzumab Fab (pL0597, 0.5 nM kd), (2) Fab variant #1 (pL2837, 68 nM Kd), (3) Fab variant #2 (pL2921, 298 nM Kd), and (4) negative control.

FIG. 4 shows that the “hits” identified by the solid-phase assay can be efficiently recovered. Of 24 putative hits identified from an imaged source plate (left) containing ˜10,000 E coli colonies representing a cytoplasmicly expressed VHH library, 22 hits were recovered as judged by the imaged signal from replicate membranes probed at high (5.0 nM, center) and low (0.5 nM, right) concentrations of antigen (Her2). The recovery efficiency is 92% (22/24).

FIG. 5 shows that the “hits” identified by the solid-phase assay at high density plating (100 million cells) can be efficiently recovered. The two images of membranes on the left top and bottom are derived from source plates that are high density (100 million) plating of E. coli expressing cell surface VHH. The image of the membrane on the right (above the line) represents a cluster of 16 patches, each patch was derived from a putative hit (bright spot) identified from the image in the left bottom source plate. The image of the membrane on the right below the line represents a cluster of 24 patches, each patch was derived from a putative hit (bright spot) identified from the image in the left top source plate. The overall recovery efficiency from high density plating is 70% (28/40).

### DETAILED DESCRIPTION

Embodiments of the present disclosure provide compositions and methods for identifying a host cell or host cells from a population of billions of cells that produces a gene product of interest. Likewise, the methods also provide for identifying a population of cells, a colony of cells, cell strains and cell clones. Exemplary cells are described herein.

As described herein, in one embodiment the present disclosure rank orders the binding affinity for all members of an E. coli library that express either a full mAb or antibody fragment, either on the cell surface or intracellularly. The described workflow has been tested to rank order the binding affinity for 100 million independent clones on a single 10 cm agar plate. In this way, the present disclosure can rank order>1 billion E. coli on a single 10 cm agar plate. Thus, the present disclosure can evaluate an E. coli antibody or antibody fragment library to determine whether a reasonable binding affinity subset exists (Dissociation constant<70 nM Kd) at the first step. If reasonable binding affinity clones are identified, the workflow allows for the recovery of such clones as pure strains.

Relative affinity is calculable using the methods and workflows provided herein. Relative affinity, as used herein, means the comparison of the signal obtained from a dose response titration of the probe.

The methods provided herein include a plating step, for example on a growth substrate. Growth substrates, for bacterial cell growth for example, are well-known in the art. In some embodiments, the growth substrate is agar (e.g., in a petri dish such as a10 cm dish). As is routinely practiced in the art, the growth conditions may optionally comprise antibiotic selection. In this way, the present methods may thus use an expression construct that includes a selectable marker. Moreover, conditions that allow host cell growth are well-known in the art and include, for example, incubation at around 37° C. or around 30° C. As described herein, each (original) plate may comprise hundreds, thousands, millions, or billions of cells.

As described herein, the present methods may use one or more replica plates. It will be appreciated that the present methods may use 2, 3, 4, 5, 6, 7, 8, 9, 10 or more replica plates. As described herein, one advantage to using multiple replica plates is the ability to determine a relative affinity value or ranking for the gene product of interest that is produced by the cells (e.g., by using variable concentrations of probe prior to imaging, as described herein). Replica plates can be prepared according to well-known techniques. In one embodiment, a membrane such as a nitrocellulose or PVDF membrane is used.

It is possible that at very high plating density (e.g., >100 million cells), most or all of the cells are removed by the membrane lift. In such a case, the hits would be recovered from a replica that was intact (not lifted onto a membrane).

As described in Dahlroth, S-L., et al. (Dahlroth, S-L., et al., Nature Protocols, 1 (1) 253-258 (2006)), a membrane-based screening method is outlined to evaluate and recover E. coli strains by the quantity of a soluble target protein produced. The method is not well suited to evaluate and recover large (100 million to >1 billion) member antibody or antibody fragment libraries and rank order the subset that binds a given antigen by relative affinity. Unlike known techniques, the present disclosure provides (1) dense plating (>1 billion cells) of the source plate in a single 10 cm agar plate, (2) replica plating the source plate to produce identical daughter plates, (3) dose response of the daughter plates in the presence of variable concentrations of the probe, (4) embedding precise positional markers in the agar and membrane to permit recovery of the hits identified, and (5) repeat cycles of the workflow if the hit strain is not a not well separated colony to obtain a pure strain.

In one embodiment, the original plate and/or replica plates and membranes(s) are marked to allow for spatial alignment and helpful for recovery of hits. The method is also useful as a diagnostic of large libraries (number of hits and affinity of each hit) without any recovery of hits. If recovery of hits is not desired, no markings are needed. Where marking is desired, any colored substrate, such as acrylic pain or ink and the link, are contemplated. Further, any dye (fluorescent or visible or trackable such as by autoradiography) can be used to mark the agar. Ideally the dye would not diffuse into the agar to cause the signal to be reduced over time. Marking the agar with a fluorescent or colored E. coli that some cell would then be transferred to the membrane during the lift is also contemplated herein. In this way, the E coli would grow back on the agar plate to allow alignment of the membrane to the agar by visual inspection.

The processing of the membrane can depend on the cellular location of the gene product of interest (e.g., a recombinant antibody or antibody fragment). For surface display or cell wall tethered expression, no lysis or fixation is, in theory, necessary. For intracellular or cytoplasmic expression, fixation (to prevent loss of the recombinant protein) and lysis (to allow probe access) would be necessary steps. Blocking may also be done as part of a workflow described herein. The blocking steps described herein reduce background noise. Both the membrane and the E. coli itself will bind to the probe in non-specific fashion. Permeabilization of the cells may also be performed, as described herein. In some embodiments, the gene product of interest may be surface display or secreted products that are tethered to the cell wall, or secreted products that can bind small probes that can pass through porins in the outer membrane. In such embodiments, permeabilization may not be necessary.

The present disclosure provides methods, in some embodiments, that include conditions that allow immobilization of cellular components and comprise contacting the membrane with a composition comprising, for example, glutaraldehyde or paraformaldehyde. Additional cell permeable cross-linkers are well known in the art (see, e.g., ThermoFisher Scientific's cross-linker selection tool). The present disclosure also provides methods, in some embodiments, that include conditions that allow permeabilization of the host cells by contacting the membrane with a composition comprising one or more of lysozyme and EDTA. Other agents for lysing the cells are well known in the art.

As described herein, probes are used to visualize “hits” or cells or strains that are sufficiently producing the gene product of interest. In some embodiments, the probe comprises a reporter moiety. For example, Fc tag, a spy tag, a Strp tag, and an Avi tag are each known and contemplated herein. In one embodiment, biotin/streptavidin-alkaline phosphatase is used to detect bound probe by chemiluminescence. The workflows described herein further contemplate that the probe is labeled directly with a chromophore or reporter enzyme. Signal amplification is also contemplated herein. Any multivalent moiety can be used for amplification. One example in the art uses bovine serum albumin with high multiples of covalently conjugated biotin. This recruits many streptavidin-alkaline phosphatase moieties. Alternatively, a biotinylated antibody can be used and is contemplated herein.

### Cells

Cells comprising one or more of the expression constructs described herein are contemplated in various embodiments of the present disclosure. Cells of the present disclosure include an outer membrane (e.g., comprised of protein and lipids) within which the fusion proteins described herein may interact or otherwise reside once they are expressed.

Prokaryotic host cells. In some embodiments of the disclosure, expression constructs designed for expression of gene products, including fusion proteins as described herein, are provided in host cells, such as prokaryotic host cells. Prokaryotic host cells can include archaea (such as Haloferax volcanii, Sulfolobus solfataricus), Gram-positive bacteria (such as Bacillus subtilis, Bacillus licheniformis, Brevibacillus choshinensis, Lactobacillus brevis, Lactobacillus buchneri, Lactococcus lactis, and Streptomyces lividans), or Gram-negative bacteria, including Alphaproteobacteria (Agrobacterium tumefaciens, Caulobacter crescentus, Rhodobacter sphaeroides, and Sinorhizobium meliloti), Betaproteobacteria (Alcaligenes eutrophus), and Gammaproteobacteria (Acinetobacter calcoaceticus, Azotobacter vinelandii, Escherichia coli, Pseudomonas aeruginosa, and Pseudomonas putida). Preferred host cells include Gammaproteobacteria of the family Enterobacteriaceae, such as Enterobacter, Erwinia, Escherichia (including E. coli), Klebsiella, Proteus, Salmonella (including Salmonella typhimurium), Serratia (including Serratia marcescans), and Shigella.

Eukaryotic host cells. Many additional types of host cells can be used for the expression systems of the present disclosure, including eukaryotic cells such as yeast (Candida shehatae, Kluyveromyces lactis, Kluyveromyces fragilis, other Kluyveromyces species, Pichia pastoris, Saccharomyces cerevisiae, Saccharomyces pastorianus also known as Saccharomyces carlsbergensis, Schizosaccharomyces pombe, Dekkera/Brettanomyces species, and Yarrowia lipolyticd); other fungi (Aspergillus nidulans, Aspergillus niger, Neurospora crassa, Penicillium, Tolypocladium, Trichoderma reesia); insect cell lines (Drosophila melanogaster Schneider 2 cells and Spodoptera frugiperda Sf9 cells); and mammalian cell lines including immortalized cell lines (Chinese hamster ovary (CHO) cells, HeLa cells, baby hamster kidney (BHK) cells, monkey kidney cells (COS), human embryonic kidney (HEK, 293, or HEK-293) cells, and human hepatocellular carcinoma cells (Hep G2)). The above host cells are available from the American Type Culture Collection.

As described in WO/2017/106583, incorporated by reference in its entirety herein, producing gene products such as therapeutic proteins at commercial scale and in soluble form is addressed by providing suitable host cells capable of growth at high cell density in fermentation culture, and which can produce soluble gene products in the oxidizing host cell cytoplasm through highly controlled inducible gene expression. Host cells of the present disclosure with these qualities are produced by combining some or all of the following characteristics. (1) The host cells are genetically modified to have an oxidizing cytoplasm, through increasing the expression or function of oxidizing polypeptides in the cytoplasm, and/or by decreasing the expression or function of reducing polypeptides in the cytoplasm. Specific examples of such genetic alterations are provided herein. Optionally, host cells can also be genetically modified to express chaperones and/or cofactors that assist in the production of the desired gene product(s), and/or to glycosylate polypeptide gene products. (2) The host cells comprise one or more expression constructs designed for the expression of one or more gene products of interest; in certain embodiments, at least one expression construct comprises an inducible promoter and a polynucleotide encoding a gene product to be expressed from the inducible promoter. (3) The host cells contain additional genetic modifications designed to improve certain aspects of gene product expression from the expression construct(s). In particular embodiments, the host cells (A) have an alteration of gene function of at least one gene encoding a transporter protein for an inducer of at least one inducible promoter, and as another example, wherein the gene encoding the transporter protein is selected from the group consisting of araE, araE, araG, araH, rhaT, xylF, xylG, and xylH, or particularly is araE, or wherein the alteration of gene function more particularly is expression of araE from a constitutive promoter; and/or (B) have a reduced level of gene function of at least one gene encoding a protein that metabolizes an inducer of at least one inducible promoter, and as further examples, wherein the gene encoding a protein that metabolizes an inducer of at least one said inducible promoter is selected from the group consisting of araA, araB, araD, prpB, prpD, rhaA, rhaB, rhaD, xylA, and xylB; and/or (C) have a reduced level of gene function of at least one gene encoding a protein involved in biosynthesis of an inducer of at least one inducible promoter, which gene in further embodiments is selected from the group consisting of scpA/sbm, argK/ygfD, scpB/ygfG, scpC/ygfH, rmlA, rmlB, rmlC, and rmlD.

Host Cells with Oxidizing Cytoplasm. The expression systems of the present disclosure are designed to express gene products; in certain embodiments of the disclosure, the gene products are expressed in a host cell. Examples of host cells are provided that allow for the efficient and cost-effective expression of gene products, including components of multimeric products. Host cells can include, in addition to isolated cells in culture, cells that are part of a multicellular organism, or cells grown within a different organism or system of organisms. In certain embodiments of the disclosure, the host cells are microbial cells such as yeasts (Saccharomyces, Schizosaccharomyces, etc.) or bacterial cells, or are gram-positive bacteria or gram-negative bacteria, or are E. coli, or are an E. coli B strain, or are E. coli (B strain) EB0001 cells (also called E. coli ASE (DGH) cells), or are E. coli (B strain) EB0002 cells. In growth experiments with E. coli host cells having oxidizing cytoplasm, specifically the E. coli B strains SHuffle® Express (NEB Catalog No. C3028H) and SHuffle® T7 Express (NEB Catalog No. C3029H) and the E. coli K strain SHuffle® T7 (NEB Catalog No. C3026H), these E. coli B strains with oxidizing cytoplasm are able to grow to much higher cell densities than the most closely corresponding E. coli K strain (WO/2017/106583).

Alterations to host cell gene functions. Certain alterations can be made to the gene functions of host cells comprising inducible expression constructs, to promote efficient and homogeneous induction of the host cell population by an inducer. Preferably, the combination of expression constructs, host cell genotype, and induction conditions results in at least 75% (more preferably at least 85%, and most preferably, at least 95%) of the cells in the culture expressing gene product from each induced promoter, as measured by the method of Khlebnikov et al. described in Example 9 of WO/2017/106583. For host cells other than E. coli, these alterations can involve the function of genes that are structurally similar to an E. coli gene, or genes that carry out a function within the host cell similar to that of the E. coli gene. Alterations to host cell gene functions include eliminating or reducing gene function by deleting the gene protein-coding sequence in its entirety, or deleting a large enough portion of the gene, inserting sequence into the gene, or otherwise altering the gene sequence so that a reduced level of functional gene product is made from that gene. Alterations to host cell gene functions also include increasing gene function by, for example, altering the native promoter to create a stronger promoter that directs a higher level of transcription of the gene, or introducing a missense mutation into the protein-coding sequence that results in a more highly active gene product. Alterations to host cell gene functions include altering gene function in any way, including for example, altering a native inducible promoter to create a promoter that is constitutively activated. In addition to alterations in gene functions for the transport and metabolism of inducers, as described herein with relation to inducible promoters, and/or an altered expression of chaperone proteins, it is also possible to alter the reduction-oxidation environment of the host cell.

Host cell reduction-oxidation environment. In bacterial cells such as E. coli, proteins that need disulfide bonds are typically exported into the periplasm where disulfide bond formation and isomerization is catalyzed by the Dsb system, comprising DsbABCD and DsbG. Increased expression of the cysteine oxidase DsbA, the disulfide isomerase DsbC, or combinations of the Dsb proteins, which are all normally transported into the periplasm, has been utilized in the expression of heterologous proteins that require disulfide bonds (Makino et al., Microb Cell Fact 2011 May 14; 10:32). It is also possible to express cytoplasmic forms of these Dsb proteins, such as a cytoplasmic version of DsbA and/or of DsbC (‘cDsbA or ‘cDsbC’), that lacks a signal peptide and therefore is not transported into the periplasm. Cytoplasmic Dsb proteins such as cDsbA and/or cDsbC are useful for making the cytoplasm of the host cell more oxidizing and thus more conducive to the formation of disulfide bonds in heterologous proteins produced in the cytoplasm. The host cell cytoplasm can also be made less reducing and thus more oxidizing by altering the thioredoxin and the glutaredoxin/glutathione enzyme systems directly: mutant strains defective in glutathione reductase (gor) or glutathione synthetase (gshB), together with thioredoxin reductase (trxB), render the cytoplasm oxidizing. These strains are unable to reduce ribonucleotides and therefore cannot grow in the absence of exogenous reductant, such as dithiothreitol (DTT). Suppressor mutations (such as ahpC* and ahpCA, Lobstein et al., Microb Cell Fact 2012 May 8; 11:56; doi: 10.1186/1475-2859-11-56) in the gene ahpC, which encodes the peroxiredoxin AhpC, convert it to a disulfide reductase that generates reduced glutathione, allowing the channeling of electrons onto the enzyme ribonucleotide reductase and enabling the cells defective in gor and trxB, or defective in gshB and trxB, to grow in the absence of DTT. A different class of mutated forms of AhpC can allow strains, defective in the activity of gamma-glutamylcysteine synthetase (gshA) and defective in trxB, to grow in the absence of DTT; these include AhpC V164G, AhpC S71F, AhpC E173/S71F, AhpC E171Ter, and AhpC dup162-169 (Faulkner et al., Proc Natl Acad Sci USA 2008 May 6; 105 (18): 6735-6740, Epub 2008 May 2). In such strains with oxidizing cytoplasm, exposed protein cysteines become readily oxidized in a process that is catalyzed by thioredoxins, in a reversal of their physiological function, resulting in the formation of disulfide bonds. Other proteins that may be helpful to reduce the oxidative stress effects in host cells of an oxidizing cytoplasm are HPI (hydroperoxidase I) catalase-peroxidase encoded by E. coli katG and HPII (hydroperoxidase II) catalase-peroxidase encoded by E. coli katE, which disproportionate peroxide into water and 02 (Farr and Kogoma, Microbiol Rev. 1991 December; 55 (4): 561-585; Review). Increasing levels of KatG and/or KatE protein in host cells through induced coexpression or through elevated levels of constitutive expression is an aspect of some embodiments of the disclosure.

Another alteration that can be made to host cells is to express the sulfhydryl oxidase Ervlp from the inner membrane space of yeast mitochondria in the host cell cytoplasm, which has been shown to increase the production of a variety of complex, disulfide-bonded proteins of eukaryotic origin in the cytoplasm of E. coli, even in the absence of mutations in gor or trxB (Nguyen et al, Microb Cell Fact 2011 Jan. 7; 10:1).

Host cells comprising expression constructs preferably also express cDsbA and/or cDsbC and/or Ervlp; are deficient in trxB gene function; are also deficient in the gene function of either gor, gshB, or gshA; optionally have increased levels of katG and/or katE gene function; and express an appropriate mutant form of AhpC so that the host cells can be grown in the absence of DTT.

Chaperones. In some embodiments, desired gene products are coexpressed with other gene products, such as chaperones, that are beneficial to the production of the desired gene product. Chaperones are proteins that assist the non-covalent folding or unfolding, and/or the assembly or disassembly, of other gene products, but do not occur in the resulting monomeric or multimeric gene product structures when the structures are performing their normal biological functions (having completed the processes of folding and/or assembly). Chaperones can be expressed from an inducible promoter or a constitutive promoter within an expression construct, or can be expressed from the host cell chromosome; preferably, expression of chaperone protein(s) in the host cell is at a sufficiently high level to produce coexpressed gene products that are properly folded and/or assembled into the desired product. Examples of chaperones present in E. coli host cells are the folding factors DnaK/DnaJ/GrpE, DsbC/DsbG, GroEL/GroES, IbpA/IbpB, Skp, Tig (trigger factor), and FkpA, which have been used to prevent protein aggregation of cytoplasmic or periplasmic proteins. DnaK/DnaJ/GrpE, GroEL/GroES, and ClpB can function synergistically in assisting protein folding and therefore expression of these chaperones in combinations has been shown to be beneficial for protein expression (Makino et al., Microb Cell Fact 2011 May 14; 10:32). When expressing eukaryotic proteins in prokaryotic host cells, a eukaryotic chaperone protein, such as protein disulfide isomerase (PDI) from the same or a related eukaryotic species, is in certain embodiments of the disclosure coexpressed or inducibly coexpressed with the desired gene product.

One chaperone that can be expressed in host cells is a protein disulfide isomerase from Humicola insolens, a soil hyphomycete (soft-rot fungus). An amino acid sequence of Humicola insolens PDI is shown as SEQ ID NO: 1 of WO/2017/106583; it lacks the signal peptide of the native protein so that it remains in the host cell cytoplasm. The nucleotide sequence encoding PDI was optimized for expression in E. coli; the expression construct for PDI is shown as SEQ ID NO: 2 of WO/2017/106583. SEQ ID NO: 2 contains a GCTAGC Nhel restriction site at its 5′ end, an AGGAGG ribosome binding site at nucleotides 7 through 12, the PDI coding sequence at nucleotides 21 through 1478, and a GTCGAC Sail restriction site at its 3′ end. The nucleotide sequence of SEQ ID NO: 2 was designed to be inserted immediately downstream of a promoter, such as an inducible promoter. The Nhel and Sail restriction sites in SEQ ID NO: 2 can be used to insert it into a vector multiple cloning site, such as that of the pSOL expression vector (SEQ ID NO: 3 of WO/2017/106583), described in published US patent application US2015353940A1, which is incorporated by reference in its entirety herein. Other PDI polypeptides can also be expressed in host cells, including PDI polypeptides from a variety of species (Saccharomyces cerevisiae (UniProtKB PI 7967), Homo sapiens (UniProtKB P07237), Mus musculus (UniProtKB P09103), Caenorhabditis elegans (UniProtKB Q 17770 and Q 17967), Arabdopsis thaliana (UniProtKB 048773, Q9XI01, Q9S G3, Q9LJU2, Q9MAU6, Q94F09, and Q9T042), Aspergillus niger (UniProtKB Q12730) and also modified forms of such PDI polypeptides. In certain embodiments of the disclosure, a PDI polypeptide expressed in host cells of the disclosure shares at least 70%, or 80%, or 90%, or 95% amino acid sequence identity across at least 50% (or at least 60%, or at least 70%, or at least 80%, or at least 90%) of the length of SEQ ID NO: 1 of WO/2017/106583, where amino acid sequence identity is determined according to Example 10 of WO/2017/106583.

Cellular transport of cofactors. When using the expression systems of the disclosure to produce enzymes that require cofactors for function, it is helpful to use a host cell capable of synthesizing the cofactor from available precursors, or taking it up from the environment. Common cofactors include ATP, coenzyme A, flavin adenine dinucleotide (FAD), NAD+/NADH, and heme. Polynucleotides encoding cofactor transport polypeptides and/or cofactor synthesizing polypeptides can be introduced into host cells, and such polypeptides can be constitutively expressed, or inducibly coexpressed with the gene products to be produced by methods of the disclosure.

Glycosylation of polypeptide gene products. Host cells can have alterations in their ability to glycosylate polypeptides. For example, eukaryotic host cells can have eliminated or reduced gene function in glycosyltransferase and/or oligo-saccharyltransferase genes, impairing the normal eukaryotic glycosylation of polypeptides to form glycoproteins. Prokaryotic host cells such as E. coli, which do not normally glycosylate polypeptides, can be altered to express a set of eukaryotic and prokaryotic genes that provide a glycosylation function (DeLisa et al., WO2009089154A2, 2009 Jul. 16).

Available host cell strains with altered gene functions. To create preferred strains of host cells to be used in the expression systems and methods of the disclosure, it is useful to start with a strain that already comprises desired genetic alterations (Table A; WO/2017/106583).

TABLE A
Exemplary host cell strains

| Strain | Genotype | Source |
|---|---|---|
| E, coli TOP10 | F-mcrA Δ(mrr-hsdRMS-mcrBC) | Invitrogen Life |
| | Φ80lacZ ΔM15 | Technologies |
| | Δlacx74 recA1 araD139 | Catalog Nos. C404-10, |
| | Δ(ara-leu)7697 galU | C4040-03, |
| | galK rpsL (Str^{R}) | C4040-06, C4040- |
| | endA1 nupG λ | 50, and C4040-52 |
| E, coli | Δ(ara-leu)7697 Δlacx74 | Merck (EMD |
| Origami ™ | ΔphoA PvuII | Millipore |
| 2 | phoR araD139 ahpC | Chemicals) |
| | gale galK rpsL | Catalog No. |
| | F'[lac⁺ lacI^{q}ª pro] | 71344 |
| | gor522::Tn10 trxB | |
| | (Str^{R}, Tet^{R}) | |
| E, coli | fhuA2 [lon] ompT ahpC | New England Biolabs |
| SHuffle ® | gal λatt::pNEB3- | Catalog No. C3028H |
| Express | rl-cDsbc (Spec, lacI) | |
| | ΔtrxB sulA11 | |
| | R(mcr-73::miniTn10-Tet^{S})2 | |
| | [dcm] R(zgb-210::Tn10-Tet^{S}) | |
| | endAl Δgor | |
| | Δ(mcrC-mrr) 114::IS10 | |

### Gene Products

As described herein, cells, cell lines and/or strains that produce one or more gene products of interest are provided. Cells, including genetically modified host cells as described herein, may express one or more gene products of interest. Gene products may include, without limitation, a nucleic acid such as a mRNA, or any protein or fragment, analog or derivative thereof. The present disclosure also provides that the screening assay described herein can be used for any target molecule (e.g., any recombinant protein or any other biomolecule or chemical entity) so long as there is a (labeled) binding partner (probe) for the target molecule and the target molecule can be immobilized to the cell or the membrane (if not expressed on the cell surface).

Proteins may include biologically active derivatives or variants or fragments. As used herein “biologically active derivative” or “biologically active variant” includes any derivative or variant of a molecule having substantially the same functional and/or biological properties of said molecule, such as binding properties, and/or the same structural basis, such as a peptidic backbone or a basic polymeric unit.

An “analog,” such as a “variant” or a “derivative,” is a compound substantially similar in structure and having the same biological activity, albeit in certain instances to a differing degree, to a naturally-occurring molecule. For example, a polypeptide variant refers to a polypeptide sharing substantially similar structure and having the same biological activity as a reference polypeptide. Variants or analogs differ in the composition of their amino acid sequences compared to the naturally-occurring polypeptide from which the analog is derived, based on one or more mutations involving (i) deletion of one or more amino acid residues at one or more termini of the polypeptide and/or one or more internal regions of the naturally-occurring polypeptide sequence (e.g., fragments), (ii) insertion or addition of one or more amino acids at one or more termini (typically an “addition” or “fusion”) of the polypeptide and/or one or more internal regions (typically an “insertion”) of the naturally-occurring polypeptide sequence or (iii) substitution of one or more amino acids for other amino acids in the naturally-occurring polypeptide sequence. By way of example, a “derivative” is a type of analog and refers to a polypeptide sharing the same or substantially similar structure as a reference polypeptide that has been modified, e.g., chemically.

A variant polypeptide is a type of analog polypeptide and includes insertion variants, wherein one or more amino acid residues are added to a biomolecule amino acid sequence of the disclosure. Insertions may be located at either or both termini of the protein, and/or may be positioned within internal regions of the therapeutic protein amino acid sequence. Insertion variants, with additional residues at either or both termini, include for example, fusion proteins and proteins including amino acid tags or other amino acid labels. In one aspect, the biomolecule optionally contains an N-terminal Met, especially when the molecule is expressed recombinantly in a bacterial cell such as E. coli. In another aspect, the biomolecule includes histidine tag (His-tag).

In deletion variants, one or more amino acid residues in a biomolecule polypeptide as described herein are removed. Deletions can be effected at one or both termini of the protein polypeptide, and/or with removal of one or more residues within the therapeutic protein amino acid sequence. Deletion variants, therefore, include fragments of a protein polypeptide sequence.

In substitution variants, one or more amino acid residues of a biomolecule are removed and replaced with alternative residues. In one aspect, the substitutions are conservative in nature and conservative substitutions of this type are well known in the art. Alternatively, the disclosure embraces substitutions that are also non-conservative. Exemplary conservative substitutions are described in Lehninger, [Biochemistry, 2nd Edition; Worth Publishers, Inc., New York (1975), pp. 71-77] and are set out immediately below.

Proteins contemplated herein include full-length proteins, precursors of full-length proteins, biologically active subunits or fragments of full length proteins, as well as biologically active derivatives and variants of any of these forms of therapeutic proteins. Thus, proteins include those that (1) have an amino acid sequence that has greater than about 60%, about 65%, about 70%, about 75%, about 80%, about 85%, about 90%, about 91%, about 92%, about 93%, about 94%, about 95%, about 96%, about 97%, about 98% or about 99% or greater amino acid sequence identity, over a region of at least about 25, about 50, about 100, about 200, about 300, about 400, or more amino acids, to a polypeptide encoded by a referenced nucleic acid or an amino acid sequence described herein. According to the present disclosure, the term “recombinant protein” includes any protein obtained via recombinant DNA technology. In certain embodiments, the term encompasses proteins as described herein.

In some embodiments, the protein is a therapeutic protein such as monoclonal or polyclonal antibody or other glycoprotein, a biosimilar, a Fc-fusion, an enzyme, a vaccine, a hormone, a cytokine, or a growth factor. In still other embodiments, gene product is an anticoagulant, a blood factor, a bone morphogenic protein, an interleukin, an interferon, a thrombolytic, or any protein produced by recombinant means. In other embodiments, the methods provide that the methods can be used with any biomolecule molecule or chemical entity, including small molecules, that are produced by cells described herein and that have a probe that can be used for binding steps.

The term “antibody” as used herein refers to whole antibodies that interact with (e.g., by binding, steric hindrance, stabilizing/destabilizing, spatial distribution) an epitope on a target antigen. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes for example, monoclonal antibodies, human antibodies, humanized antibodies, camelised antibodies, chimeric antibodies, single-chain Fvs (scFv), disulfide-linked Fvs (sdFv), Fab fragments, F (ab′) fragments, and anti-idiotypic (anti-Id) antibodies (including, e.g., anti-Id antibodies to antibodies of the invention), and epitope-binding fragments of any of the above. The antibodies can be of any isotype (e.g., IgG, IgE, IgM, IgD, IgA and IgY), class (e.g., IgG1, IgG2, IgG3, IgG4, IgA1 and IgA2) or subclass. The antibody or epitope-binding fragments may be, or be a component of, a multi-specific molecule.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminus is a variable region and at the C-terminus is a constant region; the CH3 and CL domains actually comprise the carboxy-terminus of the heavy and light chain, respectively.

The phrase “antibody fragment”, as used herein, refers to one or more portions of an antibody that retain the ability to specifically interact with (e.g., by binding, steric hindrance, stabilizing/destabilizing, spatial distribution) a target epitope. Examples of binding fragments include, but are not limited to, a Fab fragment, a monovalent fragment consisting of the VL, VH, CL and CH1 domains; a F (ab) 2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., (1989) Nature 341:544-546), which consists of a VH domain; and an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., (1988) Science 242:423-426; and Huston et al., (1988) Proc. Natl. Acad. Sci. 85:5879-5883). Such single chain antibodies are also intended to be encompassed within the term “antibody fragment”. These antibody fragments are obtained using conventional techniques known to those of skill in the art, and the fragments are screened for utility in the same manner as are intact antibodies.

The present disclosure also provides, in some embodiments, a fusion protein that includes an optional linker, e.g., a flexible linker sequence, between two protein components. In some embodiments, the linker is at least 5, at least 10, at least 15, at least 20, at least 25, at least 30, at least 35, at least 40, at least 45, or at least 50 amino acids in length. In other embodiments, the linker is less than 5, less than 10, less than 15, less than 20, less than 25, less than 30, less than 35, less than 40, less than 45, or less than 50 amino acids in length. In one embodiment, the linker is 23 amino acids in length. The linkers may be, in various embodiments, flexible, rigid, or cleavable (e.g., disulfide, protease sensitive sequences) (Chen, X., et al., Advanced Drug Delivery Reviews, 65 (1): 1357-1369 (2013)). Linkers may be derived from naturally occurring multi-domain proteins, or they may be empirical (See Argos, P., J. Mol. Biol., 211:943-958 (1990); and Heringa, G. R., Protein Eng., 15:871-879 (2002) which independently compared several properties of natural linkers, such as length, hydrophobicity, amino acid residues, and secondary structure). Threonine (Thr), serine (Ser), proline (Pro), glycine (Gly), aspartic acid (Asp), lysine (Lys), glutamine (Gln), asparagine (Asn), and alanine (Ala) are, in some embodiments, preferable linker constituents, as are arginine (Arg), phenylalanine (Phe), and glutamic acid (Glu). In general, preferable amino acids are polar uncharged or charged residues. Natural linkers adopt various secondary structures, such as helical, β-strand, coil/bend and turns, to exert their functions.

### Expression Constructs

In some embodiments of the present disclosure, inducible promoters are contemplated for use with the expression constructs. Exemplary promoters are described herein and are also described in WO/2016/205570, incorporated by reference in its entirety herein. As described herein, the cells comprising one or more expression constructs may optionally include one or more inducible promoters to express a gene product of interest. In one embodiment, the gene product is a fusion protein as described herein. In other embodiments, the gene product is a protein, for example therapeutic protein.

Expression Constructs. Expression constructs are polynucleotides designed for the expression of one or more gene products of interest, and thus are not naturally occurring molecules. Expression constructs can be integrated into a host cell chromosome, or maintained within the host cell as polynucleotide molecules replicating independently of the host cell chromosome, such as plasmids or artificial chromosomes. An example of an expression construct is a polynucleotide resulting from the insertion of one or more polynucleotide sequences into a host cell chromosome, where the inserted polynucleotide sequences alter the expression of chromosomal coding sequences. An expression vector is a plasmid expression construct specifically used for the expression of one or more gene products. One or more expression constructs can be integrated into a host cell chromosome or be maintained on an extrachromosomal polynucleotide such as a plasmid or artificial chromosome. The following are descriptions of particular types of polynucleotide sequences that can be used in expression constructs for the expression or coexpression of gene products, including fusion proteins as described herein.

Origins of replication. Expression constructs must comprise an origin of replication, also called a replicon, in order to be maintained within the host cell as independently replicating polynucleotides. Different replicons that use the same mechanism for replication cannot be maintained together in a single host cell through repeated cell divisions. As a result, plasmids can be categorized into incompatibility groups depending on the origin of replication that they contain, as shown in Table 2 of WO/2016/205570. Origins of replication can be selected for use in expression constructs on the basis of incompatibility group, copy number, and/or host range, among other criteria. As described above, if two or more different expression constructs are to be used in the same host cell for the coexpression of multiple gene products, it is best if the different expression constructs contain origins of replication from different incompatibility groups: a pMBl replicon in one expression construct and a p15A replicon in another, for example. The average number of copies of an expression construct in the cell, relative to the number of host chromosome molecules, is determined by the origin of replication contained in that expression construct. Copy number can range from a few copies per cell to several hundred (Table 2 of WO/2016/205570). In one embodiment of the disclosure, different expression constructs are used which comprise inducible promoters that are activated by the same inducer, but which have different origins of replication. By selecting origins of replication that maintain each different expression construct at a certain approximate copy number in the cell, it is possible to adjust the levels of overall production of a gene product expressed from one expression construct, relative to another gene product expressed from a different expression construct. As an example, to coexpress subunits A and B of a multimeric protein, an expression construct is created which comprises the colEl replicon, the am promoter, and a coding sequence for subunit A expressed from the am promoter: ‘colEl-Para-A.

Another expression construct is created comprising the pl 5A replicon, the am promoter, and a coding sequence for subunit B: ‘p15A-Para-B’. These two expression constructs can be maintained together in the same host cells, and expression of both subunits A and B is induced by the addition of one inducer, arabinose, to the growth medium. If the expression level of subunit A needed to be significantly increased relative to the expression level of subunit B, in order to bring the stoichiometric ratio of the expressed amounts of the two subunits closer to a desired ratio, for example, a new expression construct for subunit A could be created, having a modified pMB 1 replicon as is found in the origin of replication of the pUC9 plasmid (‘pUC9ori’): pUC9ori-Para-A. Expressing subunit A from a high-copy-number expression construct such as pUC9ori-Para-A should increase the amount of subunit A produced relative to expression of subunit B from p15A-Para-B. In a similar fashion, use of an origin of replication that maintains expression constructs at a lower copy number, such as pSOOl (WO/2016/205570), could reduce the overall level of a gene product expressed from that construct. Selection of an origin of replication can also determine which host cells can maintain an expression construct comprising that replicon. For example, expression constructs comprising the colEl origin of replication have a relatively narrow range of available hosts, species within the Enterobacteriaceae family, while expression constructs comprising the RK2 replicon can be maintained in E. coli, Pseudomonas aeruginosa, Pseudomonas putida, Azotobacter vinelandii, and Alcaligenes eutrophus, and if an expression construct comprises the RK2 replicon and some regulator genes from the RK2 plasmid, it can be maintained in host cells as diverse as Sinorhizobium meliloti, Agrobacterium tumefaciens, Caulobacter crescentus, Acinetobacter calcoaceticus, and Rhodobacter sphaeroides (Kiies and Stahl, Microbiol Rev 1989 December; 53 (4): 491-516).

Similar considerations can be employed to create expression constructs for inducible expression or coexpression in eukaryotic cells. For example, the 2-micron circle plasmid of Saccharomyces cerevisiae is compatible with plasmids from other yeast strains, such as pSRl (ATCC Deposit Nos. 48233 and 66069; Araki et al., J Mol Biol 1985 Mar. 20; 182 (2): 191-203) and pKDl (ATCC Deposit No. 37519; Chen et al, Nucleic Acids Res 1986 Jun. 11; 14 (11): 4471-4481).

Selectable markers. Expression constructs usually comprise a selection gene, also termed a selectable marker, which encodes a protein necessary for the survival or growth of host cells in a selective culture medium. Host cells not containing the expression construct comprising the selection gene will not survive in the culture medium. Typical selection genes encode proteins that confer resistance to antibiotics or other toxins, or that complement auxotrophic deficiencies of the host cell. One example of a selection scheme utilizes a drug such as an antibiotic to arrest growth of a host cell. Those cells that contain an expression construct comprising the selectable marker produce a protein conferring drug resistance and survive the selection regimen. Some examples of antibiotics that are commonly used for the selection of selectable markers (and abbreviations indicating genes that provide antibiotic resistance phenotypes) are: ampicillin (AmpR), chloramphenicol (CmlR or CmR), kanamycin (KanR), spectinomycin (SpcR), streptomycin (StrR), and tetracycline (TetR). Many of the plasmids in Table 2 of WO/2016/205570 comprise selectable markers, such as pBR322 (AmpR, TetR); pMOB45 (CmR, TetR); pACYClW (AmpR, KanR); and pGBMl (SpcR, StrR). The native promoter region for a selection gene is usually included, along with the coding sequence for its gene product, as part of a selectable marker portion of an expression construct. Alternatively, the coding sequence for the selection gene can be expressed from a constitutive promoter.

In various aspects, suitable selectable markers include, but are not limited to, neomycin phosphotransferase (npt II), hygromycin phosphotransferase (hpt), dihydrofolate reductase (dhfr), zeocin, phleomycin, bleomycin resistance gene (ble), gentamycin acetyltransferase, streptomycin phosphotransferase, mutant form of acetolactate synthase (als), bromoxynil nitrilase, phosphinothricin acetyl transferase (bar), enolpyruvylshikimate-3-phosphate (EPSP) synthase (aro A), muscle specific tyrosine kinase receptor molecule (MuSK-R), copper-zinc superoxide dismutase (sod1), metallothioneins (cup1, MT1), beta-lactamase (BLA), puromycin N-acetyl-transferase (pac), blasticidin acetyl transferase (bls), blasticidin deaminase (bsr), histidinol dehydrogenase (HDH), N-succinyl-5-aminoimidazole-4-carboxamide ribotide (SAICAR) synthetase (ade1), argininosuccinate lyase (arg4), beta-isopropylmalate dehydrogenase (leu2), invertase (suc2), orotidine-5′-phosphate (OMP) decarboxylase (ura3), and orthologs of any of the foregoing.

Inducible promoter. As described herein, there are several different inducible promoters that can be included in expression constructs as part of the inducible coexpression systems of the disclosure. Preferred inducible promoters share at least 80% polynucleotide sequence identity (more preferably, at least 90% identity, and most preferably, at least 95% identity) to at least 30 (more preferably, at least 40, and most preferably, at least 50) contiguous bases of a promoter polynucleotide sequence as defined in Table 1 of WO/2016/205570 by reference to the E. coli K-12 substrain MG1655 genomic sequence, where percent polynucleotide sequence identity is determined using the methods of Example 11 of WO/2016/205570. Under ‘standard’ inducing conditions (see Example 5 of WO/2016/205570), preferred inducible promoters have at least 75% (more preferably, at least 100%, and most preferably, at least 110%) of the strength of the corresponding ‘wild-type’ inducible promoter of E. coli K-12 substrain MG1655, as determined using the quantitative PCR method of De Mey et al. (Example 6 of WO/2016/205570). Within the expression construct, an inducible promoter is placed 5′ to (or ‘upstream of) the coding sequence for the gene product that is to be inducibly expressed, so that the presence of the inducible promoter will direct transcription of the gene product coding sequence in a 5′ to 3′ direction relative to the coding strand of the polynucleotide encoding the gene product.

Ribosome binding site. For polypeptide gene products, the nucleotide sequence of the region between the transcription initiation site and the initiation codon of the coding sequence of the gene product that is to be inducibly expressed corresponds to the 5′ untranslated region (‘UTR’) of the mRNA for the polypeptide gene product. Preferably, the region of the expression construct that corresponds to the 5′ UT comprises a polynucleotide sequence similar to the consensus ribosome binding site (RBS, also called the Shine-Dalgarno sequence) that is found in the species of the host cell. In prokaryotes (archaea and bacteria), the RBS consensus sequence is GGAGG or GGAGGU, and in bacteria such as E. coli, the RBS consensus sequence is AGGAGG or AGGAGGU. The RBS is typically separated from the initiation codon by 5 to 10 intervening nucleotides. In expression constructs, the RBS sequence is preferably at least 55% identical to the AGGAGGU consensus sequence, more preferably at least 70% identical, and most preferably at least 85% identical, and is separated from the initiation codon by 5 to 10 intervening nucleotides, more preferably by 6 to 9 intervening nucleotides, and most preferably by 6 or 7 intervening nucleotides. The ability of a given RBS to produce a desirable translation initiation rate can be calculated at the website salis.psu.edu/software/RBSLibraryCalculatorSearchMode, using the RBS Calculator; the same tool can be used to optimize a synthetic RBS for a translation rate across a 100,000+ fold range (Salis, Methods Enzymol 2011; 498:19-42).

Multiple cloning site. A multiple cloning site (MCS), also called a polylinker, is a polynucleotide that contains multiple restriction sites in close proximity to or overlapping each other. The restriction sites in the MCS typically occur once within the MCS sequence, and preferably do not occur within the rest of the plasmid or other polynucleotide construct, allowing restriction enzymes to cut the plasmid or other polynucleotide construct only within the MCS. Examples of MCS sequences are those in the pBAD series of expression vectors, including pBAD18, pBAD18-Cm, pBAD18-Kan, pBAD24, pBAD28, pBAD30, and pBAD33 (Guzman et al., J Bacteriol 1995 July; 177 (14): 4121-4130); or those in the pPRO series of expression vectors derived from the pBAD vectors, such as pPR018, pPR018-Cm, pPR018-Kan, pPR024, pPRO30, and pPR033 (U.S. Pat. No. 8,178,338 B2; May 15, 2012; Keasling, Jay). A multiple cloning site can be used in the creation of an expression construct: by placing a multiple cloning site 3′ to (or downstream of) a promoter sequence, the MCS can be used to insert the coding sequence for a gene product to be expressed or coexpressed into the construct, in the proper location relative to the promoter so that transcription of the coding sequence will occur. Depending on which restriction enzymes are used to cut within the MCS, there may be some part of the MCS sequence remaining within the expression construct after the coding sequence or other polynucleotide sequence is inserted into the expression construct. Any remaining MCS sequence can be upstream or, or downstream of, or on both sides of the inserted sequence. A ribosome binding site can be placed upstream of the MCS, preferably immediately adjacent to or separated from the MCS by only a few nucleotides, in which case the RBS would be upstream of any coding sequence inserted into the MCS. Another alternative is to include a ribosome binding site within the MCS, in which case the choice of restriction enzymes used to cut within the MCS will determine whether the RBS is retained, and in what relation to, the inserted sequences. A further alternative is to include a RBS within the polynucleotide sequence that is to be inserted into the expression construct at the MCS, preferably in the proper relation to any coding sequences to stimulate initiation of translation from the transcribed messenger RNA.

Expression from constitutive promoters. Expression constructs of the disclosure can also comprise coding sequences that are expressed from constitutive promoters. Unlike inducible promoters, constitutive promoters initiate continual gene product production under most growth conditions. One example of a constitutive promoter is that of the Tn3 bla gene, which encodes beta-lactamase and is responsible for the ampicillin-resistance (AmpR) phenotype conferred on the host cell by many plasmids, including pBR322 (ATCC 31344), pACYClW (ATCC 37031), and pBAD24 (ATCC 87399). Another constitutive promoter that can be used in expression constructs is the promoter for the E. coli lipoprotein gene, Ipp, which is located at positions 1755731-1755406 (plus strand) in E. coli K-12 substrain MG1655 (Inouye and Inouye, Nucleic Acids Res 1985 May 10; 13 (9): 3101-3110). A further example of a constitutive promoter that has been used for heterologous gene expression in E. coli is the trpLEDCBA promoter, located at positions 1321169-1321133 (minus strand) in E. coli K-12 substrain MG1655 (Windass et al., Nucleic Acids Res 1982 Nov. 11; 10 (21): 6639-6657). Constitutive promoters can be used in expression constructs for the expression of selectable markers, as described herein, and also for the constitutive expression of other gene products useful for the coexpression of the desired product. For example, transcriptional regulators of the inducible promoters, such as AraC, PrpR, RhaR, and XylR, if not expressed from a bidirectional inducible promoter, can alternatively be expressed from a constitutive promoter, on either the same expression construct as the inducible promoter they regulate, or a different expression construct. Similarly, gene products useful for the production or transport of the inducer, such as PrpEC, AraE, or Rha, or proteins that modify the reduction-oxidation environment of the cell, as a few examples, can be expressed from a constitutive promoter within an expression construct. Gene products useful for the production of coexpressed gene products, and the resulting desired product, also include chaperone proteins, cofactor transporters, etc.

Signal Peptides. Polypeptide gene products expressed or coexpressed by the methods of the disclosure can contain signal peptides or lack them, depending on whether it is desirable for such gene products to be exported from the host cell cytoplasm into the periplasm, or to be retained in the cytoplasm, respectively. Signal peptides (also termed signal sequences, leader sequences, or leader peptides) are characterized structurally by a stretch of hydrophobic amino acids, approximately five to twenty amino acids long and often around ten to fifteen amino acids in length, that has a tendency to form a single alpha-helix. This hydrophobic stretch is often immediately preceded by a shorter stretch enriched in positively charged amino acids (particularly lysine). Signal peptides that are to be cleaved from the mature polypeptide typically end in a stretch of amino acids that is recognized and cleaved by signal peptidase. Signal peptides can be characterized functionally by the ability to direct transport of a polypeptide, either co-translationally or post-translationally, through the plasma membrane of prokaryotes (or the inner membrane of gram negative bacteria like E. coli), or into the endoplasmic reticulum of eukaryotic cells. The degree to which a signal peptide enables a polypeptide to be transported into the periplasmic space of a host cell like E. coli, for example, can be determined by separating periplasmic proteins from proteins retained in the cytoplasm, using a method such as described in Example 12 of WO/2016/205570.

The following is a description of inducible promoters that can be used in expression constructs for expression or coexpression of gene products, along with some of the genetic modifications that can be made to host cells that contain such expression constructs. Examples of these inducible promoters and related genes are, unless otherwise specified, from Escherichia coli (E. coli) strain MG1655 (American Type Culture Collection deposit ATCC 700926), which is a substrain of E. coli K-12 (American Type Culture Collection deposit ATCC 10798). Table 1 of WO/2016/205570 lists the genomic locations, in E. coli MG1655, of the nucleotide sequences for these examples of inducible promoters and related genes. Nucleotide and other genetic sequences, referenced by genomic location as in Table 1 of WO/2016/205570, are expressly incorporated by reference herein. Additional information about E. coli promoters, genes, and strains described herein can be found in many public sources, including the online EcoliWiki resource, located at ecoliwiki.net.

Arabinose promoter. (As used herein, ‘arabinose’ means L-arabinose.) Several E. coli operons involved in arabinose utilization are inducible by arabinose—araBAD, araC, arciE, and araFGH—but the terms ‘arabinose promoter’ and ‘ara promoter’ are typically used to designate the araBAD promoter. Several additional terms have been used to indicate the E. coli araBAD promoter, such as Para, ParaB, ParaBAD, and PBAD—The use herein of ‘ara promoter’ or any of the alternative terms given above, means the E. coli araBAD promoter. As can be seen from the use of another term, ‘araC-araBAD promoter’, the araBAD promoter is considered to be part of a bidirectional promoter, with the araBAD promoter controlling expression of the araBAD operon in one direction, and the araC promoter, in close proximity to and on the opposite strand from the araBAD promoter, controlling expression of the araC coding sequence in the other direction. The AraC protein is both a positive and a negative transcriptional regulator of the araBAD promoter. In the absence of arabinose, the AraC protein represses transcription from PBAD, but in the presence of arabinose, the AraC protein, which alters its conformation upon binding arabinose, becomes a positive regulatory element that allows transcription from PBAD—The araBAD operon encodes proteins that metabolize L-arabinose by converting it, through the intermediates L-ribulose and L-ribulose-phosphate, to D-xylulose-5-phosphate. For the purpose of maximizing induction of expression from an arabinose-inducible promoter, it is useful to eliminate or reduce the function of AraA, which catalyzes the conversion of L-arabinose to L-ribulose, and optionally to eliminate or reduce the function of at least one of AraB and AraD, as well. Eliminating or reducing the ability of host cells to decrease the effective concentration of arabinose in the cell, by eliminating or reducing the cell's ability to convert arabinose to other sugars, allows more arabinose to be available for induction of the arabinose-inducible promoter. The genes encoding the transporters which move arabinose into the host cell are araE, which encodes the low-affinity L-arabinose proton symporter, and the araFGH operon, which encodes the subunits of an ABC superfamily high-affinity L-arabinose transporter. Other proteins which can transport L-arabinose into the cell are certain mutants of the LacY lactose permease: the LacY (AlWC) and the LacY (AlWV) proteins, having a cysteine or a valine amino acid instead of alanine at position 177, respectively (Morgan-Kiss et al., Proc Natl Acad Sci USA 2002 May 28; 99 (11): 7373-7377). In order to achieve homogenous induction of an arabinose-inducible promoter, it is useful to make transport of arabinose into the cell independent of regulation by arabinose. This can be accomplished by eliminating or reducing the activity of the AraFGH transporter proteins and altering the expression of araE so that it is only transcribed from a constitutive promoter. Constitutive expression of araE can be accomplished by eliminating or reducing the function of the native araE gene, and introducing into the cell an expression construct which includes a coding sequence for the AraE protein expressed from a constitutive promoter. Alternatively, in a cell lacking AraFGH function, the promoter controlling expression of the host cell's chromosomal araE gene can be changed from an arabinose-inducible promoter to a constitutive promoter. In similar manner, as additional alternatives for homogenous induction of an arabinose-inducible promoter, a host cell that lacks AraE function can have any functional AraFGH coding sequence present in the cell expressed from a constitutive promoter. As another alternative, it is possible to express both the araE gene and the araFGH operon from constitutive promoters, by replacing the native araE and araFGH promoters with constitutive promoters in the host chromosome. It is also possible to eliminate or reduce the activity of both the AraE and the AraFGH arabinose transporters, and in that situation to use a mutation in the LacY lactose permease that allows this protein to transport arabinose. Since expression of the lacY gene is not normally regulated by arabinose, use of a LacY mutant such as LacY (A177C) or LacY (A177V), will not lead to the ‘all or none’ induction phenomenon when the arabinose-inducible promoter is induced by the presence of arabinose. Because the LacY (A177C) protein appears to be more effective in transporting arabinose into the cell, use of polynucleotides encoding the LacY (A177C) protein is preferred to the use of polynucleotides encoding the LacY (A177V) protein.

Propionate promoter. The ‘propionate promoter’ or ‘prp promoter’ is the promoter for the E. coli prpBCDE operon, and is also called PPΦB. Like the ara promoter, the prp promoter is part of a bidirectional promoter, controlling expression of the prpBCDE operon in one direction, and with the prpR promoter controlling expression of the prpR coding sequence in the other direction. The PrpR protein is the transcriptional regulator of the prp promoter, and activates transcription from the prp promoter when the PrpR protein binds 2-methylcitrate (‘2-MC’). Propionate (also called propanoate) is the ion, CH3CH2COO—, of propionic acid (or ‘propanoic acid’), and is the smallest of the ‘fatty’ acids having the general formula H(CH2), COOH that shares certain properties of this class of molecules: producing an oily layer when salted out of water and having a soapy potassium salt. Commercially available propionate is generally sold as a monovalent cation salt of propionic acid, such as sodium propionate (CH3CH2COONa), or as a divalent cation salt, such as calcium propionate (Ca(CH3CH2COO)2). Propionate is membrane-permeable and is metabolized to 2-MC by conversion of propionate to propionyl-CoA by PrpE (propionyl-CoA synthetase), and then conversion of propionyl-CoA to 2-MC by PrpC (2-methylcitrate synthase). The other proteins encoded by the prpBCDE operon, PrpD (2-methylcitrate dehydratase) and PrpB (2-methylisocitrate lyase), are involved in further catabolism of 2-MC into smaller products such as pyruvate and succinate. In order to maximize induction of a propionate-inducible promoter by propionate added to the cell growth medium, it is therefore desirable to have a host cell with PrpC and PrpE activity, to convert propionate into 2-MC, but also having eliminated or reduced PrpD activity, and optionally eliminated or reduced PrpB activity as well, to prevent 2-MC from being metabolized. Another operon encoding proteins involved in 2-MC biosynthesis is the scpA-argK-scpBC operon, also called the sbm-yg/DGH operon. These genes encode proteins required for the conversion of succinate to propionyl-CoA, which can then be converted to 2-MC by PrpC. Elimination or reduction of the function of these proteins would remove a parallel pathway for the production of the 2-MC inducer, and thus might reduce background levels of expression of a propionate-inducible promoter, and increase sensitivity of the propionate-inducible promoter to exogenously supplied propionate. It has been found that a deletion of sbm-ygfD-ygfG-ygfH-ygfl, introduced into E. coli BL21 (DE3) to create strain JSB (Lee and Keasling, “A propionate-inducible expression system for enteric bacteria”, Appl Environ Microbiol 2005 November; 71 (11): 6856-6862), was helpful in reducing background expression in the absence of exogenously supplied inducer, but this deletion also reduced overall expression from the prp promoter in strain JSB. It should be noted, however, that the deletion sbm-ygfD-ygfG-ygfH-ygfl also apparently affects ygfl, which encodes a putative LysR-family transcriptional regulator of unknown function. The genes sbm-yg/DGH are transcribed as one operon, and ygfl is transcribed from the opposite strand. The 3′ ends of the ygfti and ygfl coding sequences overlap by a few base pairs, so a deletion that takes out all of the sbm-yg/DGH operon apparently takes out ygfl coding function as well. Eliminating or reducing the function of a subset of the sbm-ygfDGH gene products, such as YgfG (also called ScpB, methylmalonyl-CoA decarboxylase), or deleting the majority of the sbm-yg/DGH (or scpA-argK-scpBC) operon while leaving enough of the 3′ end of the ygfli (or scpC) gene so that the expression of ygfl is not affected, could be sufficient to reduce background expression from a propionate-inducible promoter without reducing the maximal level of induced expression.

Rhamnose promoter. (As used herein, ‘rhamnose’ means L-rhamnose.) The ‘rhamnose promoter’ or ‘rha promoter’, or PrhaSR, is the promoter for the E. coli rhaSR operon. Like the ara and prp promoters, the rha promoter is part of a bidirectional promoter, controlling expression of the rhaSR operon in one direction, and with the rhaBAD promoter controlling expression of the rhaBAD operon in the other direction. The rha promoter, however, has two transcriptional regulators involved in modulating expression: RhaR and RhaS. The RhaR protein activates expression of the rhaSR operon in the presence of rhamnose, while RhaS protein activates expression of the L-rhamnose catabolic and transport operons, rhaBAD and rhaT, respectively (Wickstrum et al, J Bacteriol 2010 January; 192 (1): 225-232). Although the RhaS protein can also activate expression of the rhaSR operon, in effect RhaS negatively autoregulates this expression by interfering with the ability of the cyclic AMP receptor protein (CRP) to coactivate expression with RhaR to a much greater level. The rhaBAD operon encodes the rhamnose catabolic proteins RhaA (L-rhamnose isomerase), which converts L-rhamnose to L-rhamnulose; RhaB (rhamnulokinase), which phosphorylates L-rhamnulose to form L-rhamnulose-1-P; and RhaD (rhamnulose-1-phosphate aldolase), which converts L-rhamnulose-1-P to L-lactaldehyde and DHAP (dihydroxy acetone phosphate). To maximize the amount of rhamnose in the cell available for induction of expression from a rhamnose-inducible promoter, it is desirable to reduce the amount of rhamnose that is broken down by catalysis, by eliminating or reducing the function of RhaA, or optionally of RhaA and at least one of RhaB and RhaD. E. coli cells can also synthesize L-rhamnose from alpha-D-glucose-1-P through the activities of the proteins RmlA, RmlB, RmlC, and RmlD (also called RfbA, RfbB, RfbC, and RfbD, respectively) encoded by the rmlBDACX (or rfbBDACX) operon. To reduce background expression from a rhamnose-inducible promoter, and to enhance the sensitivity of induction of the rhamnose-inducible promoter by exogenously supplied rhamnose, it could be useful to eliminate or reduce the function of one or more of the RmlA, RmlB, RmlC, and

RmlD proteins. L-rhamnose is transported into the cell by RhaT, the rhamnose permease or L-rhamnose: proton symporter. As noted above, the expression of RhaT is activated by the transcriptional regulator RhaS. To make expression of RhaT independent of induction by rhamnose (which induces expression of RhaS), the host cell can be altered so that all functional RhaT coding sequences in the cell are expressed from constitutive promoters. Additionally, the coding sequences for RhaS can be deleted or inactivated, so that no functional RhaS is produced. By eliminating or reducing the function of RhaS in the cell, the level of expression from the rhaSR promoter is increased due to the absence of negative autoregulation by RhaS, and the level of expression of the rhamnose catalytic operon rhaBAD is decreased, further increasing the ability of rhamnose to induce expression from the rha promoter.

Xylose promoter. (As used herein, ‘xylose’ means D-xylose.) The xylose promoter, or ‘xyl promoter’, or PxyiA, means the promoter for the E. coli xylAB operon. The xylose promoter region is similar in organization to other inducible promoters in that the xylAB operon and the xylFGHR operon are both expressed from adjacent xylose-inducible promoters in opposite directions on the E. coli chromosome (Song and Park, J Bacteriol. 1997 November; 179 (22): 7025-7032). The transcriptional regulator of both the PxyiA and PxyiF promoters is XylR, which activates expression of these promoters in the presence of xylose. The xylR gene is expressed either as part of the xylFGHR operon or from its own weak promoter, which is not inducible by xylose, located between the xylH and xylR protein-coding sequences. D-xylose is catabolized by XylA (D-xylose isomerase), which converts D-xylose to D-xylulose, which is then phosphorylated by XylB (xylulokinase) to form D-xylulose-5-P. To maximize the amount of xylose in the cell available for induction of expression from a xylose-inducible promoter, it is desirable to reduce the amount of xylose that is broken down by catalysis, by eliminating or reducing the function of at least XylA, or optionally of both XylA and XylB. The xylFGHR operon encodes XylF, XylG, and XylH, the subunits of an ABC super-family high-affinity D-xylose transporter. The xylE gene, which encodes the E. coli low-affinity xylose-proton symporter, represents a separate operon, the expression of which is also inducible by xylose. To make expression of a xylose transporter independent of induction by xylose, the host cell can be altered so that all functional xylose transporters are expressed from constitutive promoters. For example, the xylFGHR operon could be altered so that the xylFGH coding sequences are deleted, leaving XylR as the only active protein expressed from the xylose-inducible PxyiF promoter, and with the xylE coding sequence expressed from a constitutive promoter rather than its native promoter. As another example, the xylR coding sequence is expressed from the PxyiA or the promoter in an expression construct, while either the xylFGHR operon is deleted and xylE is constitutively expressed, or alternatively an xylFGH operon (lacking the xylR coding sequence since that is present in an expression construct) is expressed from a constitutive promoter and the xylE coding sequence is deleted or altered so that it does not produce an active protein.

Lactose promoter. The term ‘lactose promoter’ refers to the lactose-inducible promoter for the lacZYA operon, a promoter which is also called lacZpl; this lactose promoter is located at ca. 365603-365568 (minus strand, with the NA polymerase binding (‘−35’) site at ca. 365603-365598, the Pribnow box (‘−10’) at 365579-365573, and a transcription initiation site at 365567) in the genomic sequence of the E. coli K-12 substrain MG1655 (NCBI Reference Sequence NC 000913.2, 11 Jan. 2012). In some embodiments, inducible coexpression systems of the disclosure can comprise a lactose-inducible promoter such as the lacZYA promoter. In other embodiments, the inducible coexpression systems of the disclosure comprise one or more inducible promoters that are not lactose-inducible promoters.

Alkaline phosphatase promoter. The terms ‘alkaline phosphatase promoter’ and ‘phoA promoter’ refer to the promoter for the phoApsiF operon, a promoter which is induced under conditions of phosphate starvation. The phoA promoter region is located at ca. 401647-401746 (plus strand, with the Pribnow box (‘−10’) at 401695-401701 (Kikuchi et al., Nucleic Acids Res 1981 Nov. 11; 9 (21): 5671-5678)) in the genomic sequence of the E. coli K-12 substrain MG1655 (NCBI Reference Sequence NC 000913.3, 16 Dec. 2014). The transcriptional activator for the phoA promoter is PhoB, a transcriptional regulator that, along with the sensor protein PhoR, forms a two-component signal transduction system in E. coli. PhoB and PhoR are transcribed from the phoBR operon, located at ca. 417050-419300 (plus strand, with the PhoB coding sequence at 417, 142-417,831 and the PhoR coding sequence at 417,889-419,184) in the genomic sequence of the E. coli K-12 substrain MG1655 (NCBI Reference Sequence NC 000913.3, 16 Dec. 2014). The phoA promoter differs from the inducible promoters described above in that it is induced by the lack of a substance-intracellular phosphate-rather than by the addition of an inducer. For this reason the phoA promoter is generally used to direct transcription of gene products that are to be produced at a stage when the host cells are depleted for phosphate, such as the later stages of fermentation. In some embodiments, inducible coexpression systems of the disclosure can comprise a phoA promoter. In other embodiments, the inducible coexpression systems of the disclosure comprise one or more inducible promoters that are not phoA promoters.

As described herein, it may be advantageous or desirable to remove (e.g., by way of an inducible or constitutive “curing” mechanism) an expression construct described herein, e.g., if the cell line harboring the expression construct is or will be used for commercial purposes. Thus, in some embodiments, the expression construct may comprise a “kill switch.” For example, in embodiment, the expression construct includes a temperature-sensitive origin of replication. Additional curing methods are known in the art and include using detergents and intercalating agents, drugs and antibiotics (Buckner, M. M. C., et al., FEMS Microbiology Reviews, fuy031,42, 2018, 781-804).

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a conformation switching probe” includes a plurality of such conformation switching probes and reference to “the microfluidic device” includes reference to one or more microfluidic devices and equivalents thereof known to those skilled in the art, and so forth. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible. This is intended to provide support for all such combinations.

### EXAMPLES

### Example 1

### Solid-Phase Screening on a High-Density Plate for Identifying High-Performing Bacterial Strains

Traditionally, screening methods evaluate E. coli colonies plated on a 10 cm petri dish that represent a cDNA, genomic, or expression library for clones containing the DNA sequence of interest. A replica of the plate is made with one of the two plates sacrificed for analysis. The E. coli colonies are transferred onto a solid support, such as PVDF or nitrocellulose membrane, the cells on the membrane are lysed and the probe is added to identify the desired clone(s). The probe to evaluate the E. coli library is either a labeled nucleic acid (for DNA or cDNA libraries) or an antibody (for expression libraries). Traditionally the probe is radiolabeled to permit identification by autoradiography. However, biotin-labeled probes enable the identification by streptavidin covalently linked to horse radish peroxidase or alkaline phosphatase by the hydrolysis their respective chemiluminescent substrates. The colonies that bound the probe are detected by imaging the membrane. The replica is used to recover the desired clone.

The present disclosure and Example makes the following improvements to address the shortcomings of current methodologies: (1) Multiple replicates of the source plate is made (typically three); (2) The number of cells plated at time zero can exceed 1 billion per 10 cm plate; (3) An optional signal amplification step is inserted to allow the identification of the desired clones when plated in high density or the identification of low affinity (dissociation constant ˜70 nM Kd) clones; (4) The workflow places positional landmarks in the agar and the membrane to allow for unambiguous recovery of positive clones, especially from densely plated libraries; and (5) The workflow adds additional round(s) of repeat screening when the recovered strain was from a densely plated agar where no well isolated single colonies exist.

The experiment described below was performed with three E coli strains that expressed (1) Herceptin Fab PL0597 (Kd=0.5 nM), (2) Herceptin Fab variant PL2837 (Kd=70 nM), and (3) Herceptin Fab variant PL2921 (Kd=298 nM). A three concentration dose response correctly rank ordered the affinity of the three strains by binding affinity (Kd): PL0597 (0.5 nM)<PL2837 (68 nM)<PL2921 (298 nM). Additionally, the blank control strain (AbS08019) showed low background signal.

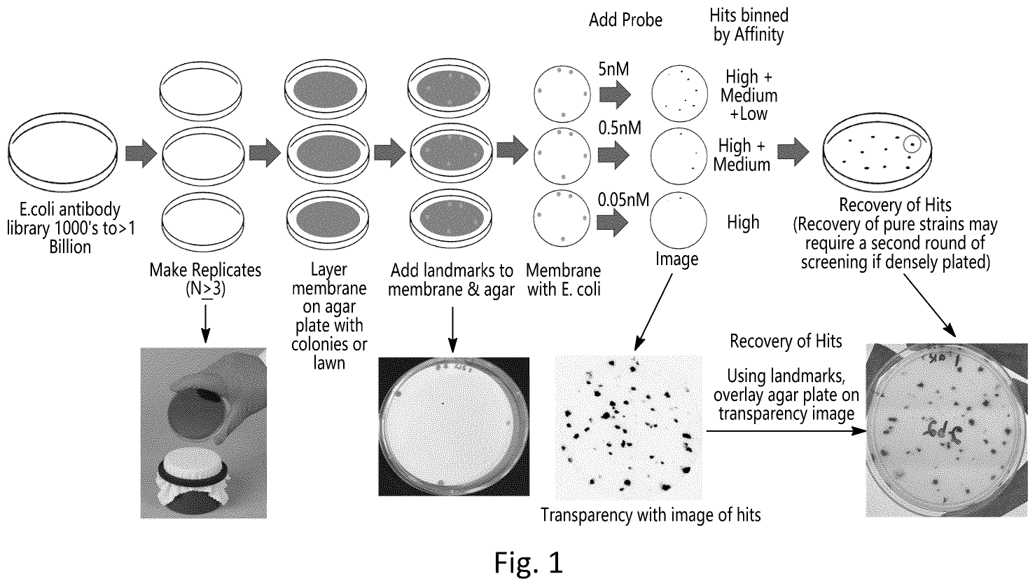

The entire screening workflow is shown in FIG. 1. A diverse E. coli expression library for either an antibody fragment (such as Fab, scFab, scFv, VHH, or nanobody) or the full antibody is plated at 5-10× multiples of the theoretical diversity, up to several billion cells at time of plating on a single 10 cm agar plate containing the appropriate antibiotics to maintain the desired plasmid(s). The plate is incubated at 37° C. overnight. The plate is replicated onto agar plates containing both inducer(s) (to induce expression of the antibody or antibody fragments) and antibiotic(s). The number of replicates is dependent on the number of bins to categorize the binding affinity for members of the library. Typically, three replicates are made for sorting the library members into three bins: low, medium, and high affinity. Additional replicas of the source plate can be prepared to further separate the high affinity variants. The replicated plates are incubated at 30° C. overnight. Next day, a nitrocellulose membrane disk is applied to the agar plate surface. While the filter is on the agar plate surface, positional landmarks are made both on the membrane and the agar as described here. The tip of a fine needle is dipped into acrylic paint. The paint coated needle is used to puncture past the membrane filter into the agar. When the needle is removed, a deposit of paint will be made in the agar at the exact location of the puncture in the membrane as well as leaving puncture marks and paint (on the air exposed side) in the filter. Five or more punctures are made around the rim of the filter, leaving behind five paint spots for positional alignment in the agar for hit recovery. When the membrane is lifted from the agar surface, most of the cells on the agar surface is removed. However, sufficient number of cells remain on the agar plate surface to permit recovery of hit(s). An optional method for recovery of hits would be to reserve one of the replicas of the source plate specifically for hit recovery. The membrane filter is placed on a Whatman paper disk with the side that contacted the agar plate facing up for a few minutes to allow the paint to dry and the excess paint to blot on the Whatman paper. Subsequently, the filtered is soaked in fixative solution containing glutaraldehyde and paraformaldehyde to immobilize cellular components. The fixative is washed away in PBS. The membrane is transferred into blocking solution (containing BSA) and incubated at 4° C. overnight with gentle rocking. The next day, the membrane is transferred to a solution containing lysozyme/EDTA for ˜5 minutes to permeabilize the cells. The permeabilized cells are exposed to the probe and incubated overnight. After extensive washing, the membrane is incubated with alkaline phosphatase substrate for 3 minutes and imaged by chemiluminescence imager.

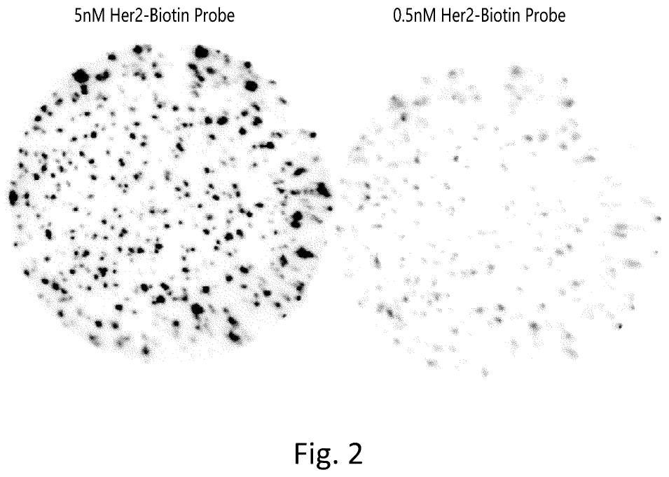

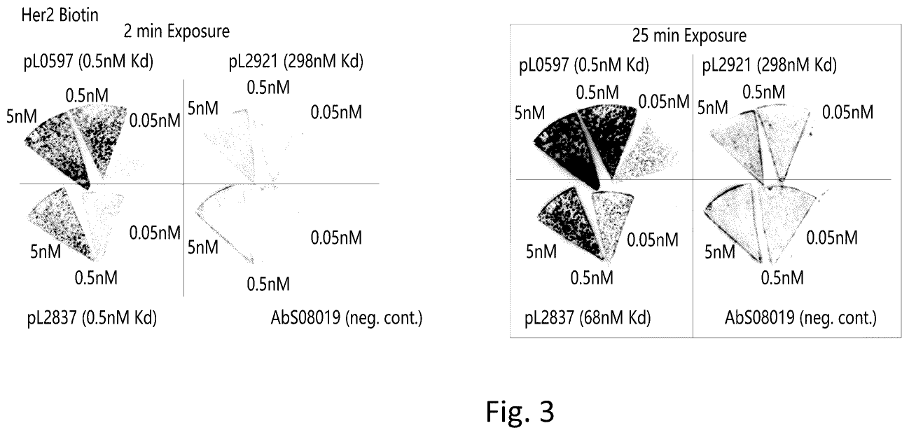

Two replicates were prepared from a source plate of ˜10,000 colonies onto plates containing the inducer. The membranes prepared from the two replica agar plates were probed with 5 nM and 0.5 nM of biotin labeled Her2 and imaged. Comparing the two membrane images (FIG. 2), these conclusions were drawn: (1) There are more dark spots (hits) in the membrane treated with 5 nM probe compared to the 0.5 nM probe; and (2) The hits that appear on both membranes have an identical relative location, that is, replica plating faithfully preserved the location of the hit strain on the original source plate to the two daughter replicas. The hits on the membrane treated with the lower concentration of probe are the strains producing the highest affinity antibody fragments. For a 3 replica plate experiment (at 5 nM, 0.5 nM and 0.05 nM probe), three replicas of the source plate would be prepared to produce three concentration dose response. The result of one version of a three replica plate experiment is provided in FIG. 3. In this case, a source plate containing a pure strain that expressed a Her2 binding Fab variant is lifted into a membrane that is subsequently divided into quadrants that then is treated with a different concentration of Her 2 probe.

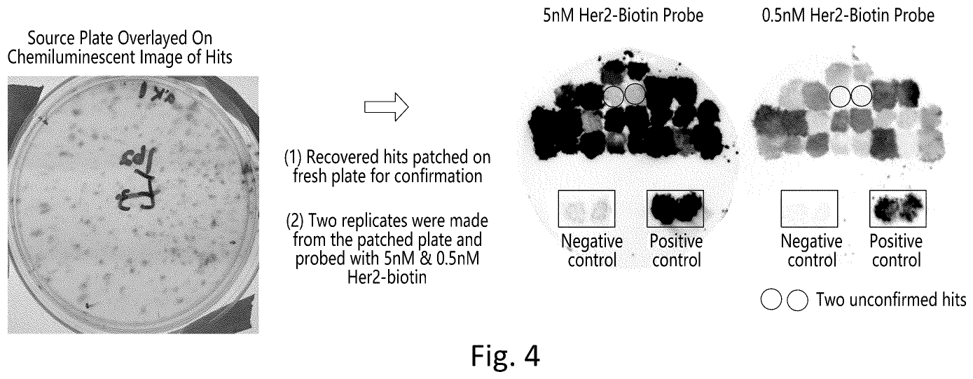

A transparency printout of the chemiluminescence image is used for the recovery of the highest affinity hits. The imager captured both the visible light image and the chemiluminescence image from the processed membrane. The location of the punctures in the membrane are captured by the visible light image and is transferred either electronically or manually to the chemiluminescence image of the filter. The chemiluminescence image is then printed onto a transparency for manual recovery of the hits from the source agar plate. As can be seen in this example, the accuracy of recovery from manually picking 24 hits was 91.7% (22 of 24 hits confirmed by analysis of the patched hits (FIG. 4).

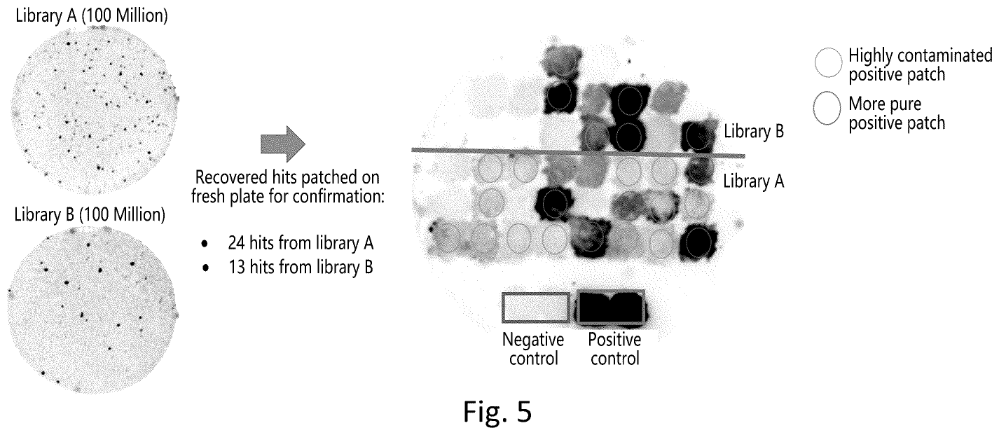

To ensure that the recovered hit is a pure strain if it was isolated from a densely plated source plate, another round of screening is performed on source plates with well isolated single colonies. In this example, the confirmed hits are pooled and serially diluted. An aliquot of each serial dilution is plated individually to ensure that the dilution that produce single colonies upon plating is covered. The identified hits from sparsely plated colonies can be easily recovered from the source as pure strains (FIG. 5).

A signal amplification step may be necessary to improve assay sensitivity in cases where the expected signal is low: (1) high density plating, (2) low affinity antibody or antibody fragment and (3) both (1) and (2) combined. Several published protocols are described in the literature for this purpose (See, e.g., ThermoFisher Scientific's biotinylated BSA product or Vector Laboratories' biotinylated anti-streptavidin product). One scheme uses biotinylated bovine serum albumin with either a monomeric streptavidin-alkaline phosphatase or a polymeric streptavidin-alkaline phosphatase dextran. One or two rounds of signal amplification should be sufficient for detection of hits with low affinity (Kd˜70 nM) antibody or antibody fragment in very high-density plating (>1 billion cells per 10 cm plate).

This Example (FIG. 5) demonstrates the performance of solid-phase Screening on a high-density plated surface displayed antibody fragment library. Approximately 100 million cells were plated on a single 10 cm agar plate. When probed with 5 nM Her2-biotin and without any signal amplification step, the imaged signal of the positive hits is very strong, indicating that detection of positive hits is possible at higher plating density of >1billion cells on a single 10 cm plate.

Additional exemplary embodiments and details of the above workflow (FIG. 1) are provided as follows:

### 1. Plating Library

Library is plated first on plates without any inducer. The plates should contain the appropriate antibiotics to enable the maintenance of the desired plasmids or genetic traits.

Spread library of cells evenly onto room temperature agar plates. Incubate overnight (16 hrs) at 30° C.

### 2. Replica Plating

Prepare appropriate number of replicas of the overnight library on to LB agar plates containing both the appropriate antibiotics and inducers.

Incubate plates at 30° C. overnight.

Inducer Solutions

| | | volume | | | Concentration | target volume | Assay conc | | amount | |
|---|---|---|---|---|---|---|---|---|---|---|
| ID | g | (L) | MW | Moles | (M) | (ml) | (uM) | fold conc | ml | media |
| Arabinose | 150.13 | 1 | 150.13 | 1.00 | 1.00 | 1000 | 250 | 4000 | 0.25 | 999.75 |
| Sodium | 96.07 | 1 | 96.07 | 1.00 | 2.00 | 1000 | 1000 | 2000 | 0.50 | 999.5 |
| propionate | | | | | | | | | | |

### 3. Transfer of Cells to Nitrocellulose Membrane

Apply one nitrocellulose membrane to each agar plate with induced cells. Make sure the membrane contacts the agar surface evenly without any wrinkles.

Dip the tip of a T pin into acrylic paint and puncture the membrane/agar at the edge. Remove T pin leaving being acrylic paint.

Repeat. Puncture the agar at approximately 5 locations around the parameter

### 4. Fixation of Cells

Transfer membrane to fixative solution with the side contacting the agar surface facing up.

Incubate membrane at room temperature for 5-10 minutes

Wash membrane 3 times with 1× phosphate buffer saline solution.

Fixative Solution:

| | stock solution | amout | target conc | | target | volume |
|---|---|---|---|---|---|---|
| Compound name | (% or mM) | (g/L) | (% or mM)) | fold | volume mL | reagent |
| Paraformaldehyde | 16 | | 2.6 | 6.2 | 40 | 6.5 |
| glutaraldehyde | 8 | | 0.04 | 200.0 | 40 | 0.2 |
| Na3PO4 pH 7.4 (mM) | 500 | | 32.25 | 15.5 | 40 | 2.58 |
| water | | | | | | 30.72 |

### 5. Blocking of Membrane

The membrane is transferred to block solution and incubate at 4 C for 16 hrs.

Block solution:

| Compound | Final | stock | | | | | |
|---|---|---|---|---|---|---|---|
| name | concentration | conc | fold | MW | 100 ml | 250 ml | 500 ml |
| Bovine Serum | 5 mg/mL | powder | | | | 1.25 g | 2.5 g |
| Albumin | | | | | | | |
| Sodium | 0.1M | powder | | 84.0007 | 0.84 g | 2.1 g | 4.2 g |
| Bicarbonate | | | | | | | |
| (NaHCO3 pH 8.6) | | | | | | | |
| EDTA | 1 mM | 500 mM | 500.0 | | 0.20 ml | 0.5 ml | 1.0 ml |
| Biotin | 100 nM | 10 mg/ml | | | 0.24 ul | 0.6 ul | 1.2 ul |

### 6. Lysis

Transfer membrane into lysis buffer. Incubate for 5-10 min at room temperature

Wash membrane 3 times in 1× PerkinElmer immunoassay buffer.

Lysis of cells:

| | | | | target | Assay | | | |
|---|---|---|---|---|---|---|---|---|
| | | | Stock | volume | concentration | | | |
| reagent name | vendor | cat no | concentration | (ml) | (nM) | fold conc | amount stock | |
| lysozyme | Millipore | 71110-5 | 20,000x | 1000 | 1x | 20,000x | 50 | ul |
| | sigma | | | | | | | |
| EDTA | VWR | 101446- | 500 mM | 1000 | 1 mM | 500x | 2 | ml |
| | | 794 | | | | | | |
| 1x | PerkinElmer | AL000F | 10x | 1000 | 1x | 10X | 100 | ml |
| PerkinElmer | | | | | | | | |
| immunoassay | | | | | | | | |
| buffer | | | | | | | | |

### 7. Biotin Block

Transfer membrane into biotin block buffer. Incubate at 4 C for 1-2 hrs.

Wash membrane with 1× PerkinElmer immunoassay buffer.

Biotin block buffer:

| | | | | target | Assay | | | | |
|---|---|---|---|---|---|---|---|---|---|
| | | | Stock | volume | concentration | | | | |
| reagent name | vendor | cat no | concentration | (ml) | (nM) | | fold conc | amount stock | |
| biotin | Millipore sigma | B4501 | 40.9 mM | 1000 | 100 | nM | 409316x | 2.4 | ul |
| EDTA | VWR | 101446-794 | 500 mM | 1000 | 1 | mM | 500x | 2 | ml |
| 1x PerkinElmer | PerkinElmer | AL000F | 10x | 1000 | 1x | | 10X | 100 | ml |
| immunoassay | | | | | | | | | |
| buffer | | | | | | | | | |

### 8. Probe

Prepare the probe solution. Rock to mix the solution at 4 C for 3-16 hrs.

Add probe to membrane. Incubate at 4 C overnight with rocking.

Probe (each membrane needs ˜15-20 ml of probe)

| | | | | | | | Concen- | Concen- | target | Assay | | |
|---|---|---|---|---|---|---|---|---|---|---|---|---|
| reagent | | cat | | volume | | | tration | tration | volume | concentration | fold | amount |
| name | vendor | no | ug | (ul) | MW | Moles | (M) | (uM) | (ul) | (nM) | conc | stock |
| Her2 | Acro | HE2- | 200 | 600 | 70200 | 2.85E−09 | 4.75E−06 | 4.748 | 20000 | 5.00 | 949.67 | 21.1 |
| biotin | biosystem | H822R | | | | | | | | | | |
| streptavid | Pierce | 21324 | 3300 | 1000 | 193000 | 1.71E−08 | 1.71E−05 | 17.098 | 20000 | 1.25 | 13678.76 | 1.5 |
| in alkaline | | | | | | | | | | | | |
| phosphatase | | | | | | | | | | | | |

### 9. Wash

Wash membrane 5 times in wash buffer:

| reagent name | vendor | cat no | comment |
|---|---|---|---|
| 1x Azure | Azure | AC2113 | dilute to 1x. Store |
| wash buffer | biosystems | | and use at room |
| | | | temperature |

### 10. Imaging

Transfer filter to a clean 10 cm petri plate. Add 2.5 ml of room temperature alkaline phosphatase substrate solution. Cover entire membrane.

Incubate at room temperature for 3 min.

Drip out excess reagent.

Place membrane in chemilumenescence imager.

Image for 2 min-5 hours.

Alkaline Phosphatase substrate:

| reagent name | vendor | cat no | comment |
|---|---|---|---|
| Alkaline Phosphatase | Sera | 5430-0055 | Store at |
| Chemilumenescent | Care | | 4-8° C. |
| substrate | | | |

Chemiluminescent imager:

| vendor | Model number |
|---|---|
| Azure Biosystems | Azure 300 |

The various embodiments described above can be combined to provide further embodiments. All U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

What is claimed:

1. A method of identifying a host cell that produces a gene product of interest, said method comprising the steps of:
(a) plating a population of host cells on a growth substrate, wherein said host cells comprise an expression construct encoding one or more gene products, and incubating the host cells under conditions that allow host cell growth on the growth substrate;
(b) preparing at least one replica plate and incubating said replica plate under conditions that allow host cell growth and production of the gene product;
(c) transferring host cells from the at the least one replica plate of (b) to a membrane;
(d) preparing the host cells that have been transferred to the membrane in (c) for probing, comprising (i) optionally fixing the host cells under conditions that allow immobilization of cellular components; (ii) blocking the host cells; and (iii) optionally lysing the host cells under conditions that allow permeabilization of the host cells;
(e) contacting the permeabilized host cells with a probe solution comprising at least one probe under conditions that allow binding of the at least one probe to a gene product of interest, and thereby forming a probe-gene product complex; and
(f) imaging the host cells under conditions that allow identifying the host cell that produces the gene product of interest.

2. The method of claim 1, wherein step (c) further comprises marking the at least one replica plate and membrane to allow spatial alignment of the at least one replica plate and membrane.

3. The method of any one of claim 1 or 2, wherein the host cell is selected from the group consisting of a eukaryotic cell, a prokaryotic cell, a bacterial cell, a mammalian cell and an insect cell.

4. The method of any of the preceding claims, wherein the gene product of interest is selected from the group consisting of a therapeutic protein, an antibody, a Fab, a scFv, a nanobody, a T cell receptor, and a chimeric antigen receptor, or fragments thereof.

5. The method of claim 4, wherein the gene product of interest is an intracellular protein.

6. The method of any of the preceding claims, wherein the population of host cells comprises approximately one thousand to approximately one billion host cells.

7. The method of any of the preceding claims, wherein the population of host cells have been modified to produce a gene product from an expression construct.

8. The method of claim 7, wherein the expression construct comprises two or more inducible promoters.

9. The method of claim 8, wherein at least one inducible promoter is a propionate-inducible promoter and at least one other inducible promoter is an L-arabinose-inducible promoter.

10. The method of any of the preceding claims, wherein the host cells have been genetically modified to comprise one or more of: (a) an alteration of gene function of at least one gene encoding a transporter protein for an inducer of at least one inducible promoter; (b) a reduced level of gene function of at least one gene encoding a protein that metabolizes an inducer of at least one inducible promoter; (c) a reduced level of gene function of at least one gene encoding a protein involved in biosynthesis of an inducer of at least one inducible promoter; (d) an altered gene function of a gene that affects the reduction/oxidation environment of the host cell cytoplasm; (e) a reduced level of gene function of a gene that encodes a reductase; (f) at least one expression construct encoding at least one disulfide bond isomerase protein; (g) at least one polynucleotide encoding a form of DsbC lacking a signal peptide; and/or (h) at least one polynucleotide encoding Ervlp.

11. The method of any of the preceding claims, wherein 2, 3, 4, 5 or more replica plates are prepared.

12. The method of claim 11, wherein the replica plates contain an antibiotic and at least one inducers of expression of the gene product of interest.

13. The method of any of the preceding claims, wherein the membrane is selected from the group consisting of a nitrocellulose membrane and a PVDF (polyvinylidene fluoride) membrane.

14. The method of any of the preceding claims, wherein the replica plate and membrane of step (c) are contacted with a colored substance.

15. The method of claim 14, wherein the colored substance is selected from the group consisting of acrylic paint and a dye.

16. The method of claim 15, wherein the colored substance is contacted to the plate and membrane with an instrument selected from the group consisting of a needle and a pen.

17. The method of any of the preceding claims, wherein the conditions that allow immobilization of cellular components comprise contacting the membrane with a composition comprising one or more of glutaraldehyde and paraformaldehyde.

18. The method of any of the preceding claims, wherein the conditions that allow permeabilization of the host cells comprise contacting the membrane with a composition comprising one or more of lysozyme and EDTA.

19. The method of any of the preceding claims, wherein the at least one probe is selected from the group consisting of an antibody or functional fragment thereof, a nucleic acid-binding protein or functional fragment thereof, a receptor or functional fragment thereof, a ligand or functional fragment thereof, and antigen or functional fragment thereof, and a peptide or polypeptide capable of being bound by the gene product of interest.

20. The method of claim 19, wherein the probe comprises a reporter moiety selected from the group consisting of biotin, a histidine tag, a Fc tag, a spy tag, a Strp tag, and an Avi tag.

21. The method of any of the preceding claims, wherein, when 2, 3, 4, 5 or more replica plates have been prepared, each membrane prepared from the replica plates is contacted with a different concentration of the probe solution.

22. The method of any of the preceding claims, wherein the imaging comprises contacting the membrane with a composition comprising an activator of the reporter moiety.

23. The method of claim 22, wherein the contacting the membrane with a composition comprising an activator of the reporter moiety is repeated once, twice, or three or more times.

24. The method of any one of claims 22-23, wherein the activator is selected from the group consisting of alkaline phosphatase, streptavidin alkaline phosphatase, and streptavidin alkaline phosphatase dextran polymer.

25. The method of any of the preceding claims, wherein the imaging comprises a method selected from the group consisting of chemiluminescence, radiography, fluorescence and colorimetric analyses.

26. The method of any of the previous claims, further comprising the step of re-plating one or more host cells that have been identified as capable of producing the gene product of interest.

27. The method of claim 26, wherein the re-plated host cell is subjected to the method of claim 1 to confirm the host cell's capability to produce the gene product of interest and/or to isolate the host cell strain from other host cell strains that have been identified as capable of producing the gene product of interest.

28. The method of any of the previous claims, further comprising determining the affinity of the probe-gene product complex.

29. A method of screening a host cell from a population of host cells that produces a gene product of interest, said method comprising the steps of:
(a) plating a population of host cells on a growth substrate, wherein said host cells comprise an expression construct encoding one or more gene products, and incubating the host cells under conditions that allow host cell growth on the growth substrate;
(b) preparing at least one replica plate and incubating said replica plate under conditions that allow host cell growth and production of the gene product;
(c) transferring host cells from the at the least one replica plate of (b) to a membrane;
(d) preparing the host cells that have been transferred to the membrane in (c) for probing, comprising (i) fixing the host cells under conditions that allow immobilization of cellular components; (ii) blocking the host cells; and (iii) optionally lysing the host cells under conditions that allow permeabilization of the host cells;
(e) contacting the permeabilized host cells with a probe solution comprising at least one probe under conditions that allow binding of the at least one probe to a gene product of interest, and thereby forming a probe-gene product complex; and
(f) imaging the host cells under conditions that allow identifying the host cell that produces the gene product of interest.

30. A method of determining the relative affinity of a probe-gene product of interest, said method comprising the steps of:
(a) plating a population of host cells on a growth substrate, wherein said host cells comprise an expression construct encoding one or more gene products, and incubating the host cells under conditions that allow host cell growth on the growth substrate;
(b) preparing at least three replica plates and incubating said replica plate under conditions that allow host cell growth and production of the gene product;
(c) transferring host cells from the at the least three replica plates of (b) to separate membranes;
(d) preparing the host cells that have been transferred to the membrane in (c) for probing, comprising (i) fixing the host cells under conditions that allow immobilization of cellular components; (ii) blocking the host cells; and (iii) optionally lysing the host cells under conditions that allow permeabilization of the host cells;
(e) contacting the permeabilized host cells on the at least three replica membranes with a probe solution comprising at least one probe under conditions that allow binding of the at least one probe to a gene product of interest, and thereby forming a probe-gene product complex, and wherein the probe solution comprises a different probe concentration; and
(f) imaging the host cells under conditions that allow determining the relative affinity of a probe-gene product of interest.
